SEQUENCE LISTING (33 sequences)

SEQ ID NO: 1 - DNA, 28 bp, Artificial Sequence
  Description of Artificial Sequence Synthetic primer
ggagtgattctctaaagagttttgtgtt

SEQ ID NO: 2 - DNA, 26 bp, Artificial Sequence
  Description of Artificial Sequence Synthetic primer
tgacattctgcattttaactatggct

SEQ ID NO: 3 - DNA, 26 bp, Artificial Sequence
  Description of Artificial Sequence Synthetic primer
tgaaattggatattgcagcagtcaga

SEQ ID NO: 4 - DNA, 31 bp, Artificial Sequence
  Description of Artificial Sequence Synthetic primer
gctcctttataagcttcttcagtatatgtca

SEQ ID NO: 5 - DNA, 30 bp, Artificial Sequence
  Description of Artificial Sequence Synthetic primer
cattttatatcctcaagccaagattaactt

SEQ ID NO: 6 - DNA, 18 bp, Artificial Sequence
  Description of Artificial Sequence Synthetic primer
gggtggtagagcaagacc

SEQ ID NO: 7 - DNA, 26 bp, Artificial Sequence
  Description of Artificial Sequence Synthetic primer
cctgcttatctgaaacttcccaactt

SEQ ID NO: 8 - DNA, 22 bp, Artificial Sequence
  Description of Artificial Sequence Synthetic primer
cctgtagtcccagctacttgga

SEQ ID NO: 9 - DNA, 20 bp, Artificial Sequence
  Description of Artificial Sequence Synthetic primer
ctcctctgtcctcccactga

SEQ ID NO: 10 - DNA, 18 bp, Artificial Sequence
  Description of Artificial Sequence Synthetic primer
gccactgcatcccatcct

SEQ ID NO: 11 - DNA, 20 bp, Artificial Sequence
  Description of Artificial Sequence Synthetic primer
agggaggcttttgagagcag

SEQ ID NO: 12 - DNA, 25 bp, Artificial Sequence
  Description of Artificial Sequence Synthetic primer
ccttgaatttcaggctcaagtctct

SEQ ID NO: 13 - DNA, 24 bp, Artificial Sequence
  Description of Artificial Sequence Synthetic primer
tagtttctacacccaagccactga

SEQ ID NO: 14 - DNA, 32 bp, Artificial Sequence
  Description of Artificial Sequence Synthetic primer
tttctaagggaaacataaaactttcattttgg

SEQ ID NO: 15 - DNA, 23 bp, Artificial Sequence
  Description of Artificial Sequence Synthetic primer
ctctttcacttggcagaacattg

SEQ ID NO: 16 - DNA, 24 bp, Artificial Sequence
  Description of Artificial Sequence Synthetic primer
gcttcgtcgaagatcagatagttg

SEQ ID NO: 17 - DNA, 28 bp, Artificial Sequence
  Description of Artificial Sequence Synthetic primer
caatgaatggcaaccagaattaaatcca

SEQ ID NO: 18 - DNA, 24 bp, Artificial Sequence
  Description of Artificial Sequence Synthetic primer
ggatcacttgagcccagaattcaa

SEQ ID NO: 19 - DNA, 22 bp, Artificial Sequence
  Description of Artificial Sequence Synthetic primer
ctttctggtcactcgcgtttac

SEQ ID NO: 20 - DNA, 23 bp, Artificial Sequence
  Description of Artificial Sequence Synthetic primer
ccatcctggtttctgaagacaca

SEQ ID NO: 21 - DNA, 23 bp, Artificial Sequence
  Description of Artificial Sequence Synthetic primer
cactgagcacatcacatttagga

SEQ ID NO: 22 - DNA, 25 bp, Artificial Sequence
  Description of Artificial Sequence Synthetic primer
gccatccagttttgttcttacaaac

SEQ ID NO: 23 - DNA, 25 bp, Artificial Sequence
  Description of Artificial Sequence Synthetic primer
gctgaattttacctcctgactccaa

SEQ ID NO: 24 - DNA, 21 bp, Artificial Sequence
  Description of Artificial Sequence Synthetic primer
cggagattgtgccattgcatt

SEQ ID NO: 25 - DNA, 24 bp, Artificial Sequence
  Description of Artificial Sequence Synthetic primer
catgcttgcaaaccactggtaaaa

SEQ ID NO: 26 - DNA, 27 bp, Artificial Sequence
  Description of Artificial Sequence Synthetic primer
ccattagtaaagaggttctgagtcgat

SEQ ID NO: 27 - DNA, 24 bp, Artificial Sequence
  Description of Artificial Sequence Synthetic primer
gagcaagacaccatctcaagaaag

SEQ ID NO: 28 - DNA, 27 bp, Artificial Sequence
  Description of Artificial Sequence Synthetic primer
gtgtatgattctctttttttccccttc

SEQ ID NO: 29 - DNA, 27 bp, Artificial Sequence
  Description of Artificial Sequence Synthetic primer
gtgtatgattctctttttttccccttt

SEQ ID NO: 30 - DNA, 18 bp, Artificial Sequence
  Description of Artificial Sequence Synthetic primer
cttccgagtgcaggtcac

SEQ ID NO: 31 - DNA, 20 bp, Artificial Sequence
  Description of Artificial Sequence Synthetic primer
ggcctgttcctcccttattt

SEQ ID NO: 32 - DNA, 11 bp, Artificial Sequence
  Description of Artificial Sequence Synthetic oligonucleotide
ggggggggggg

SEQ ID NO: 33 - DNA, 16 bp, Artificial Sequence
  Description of Artificial Sequence Synthetic oligonucleotide
tatgtatgtatgtagt

(19) United States
(12) Patent Application Publication
(10) Pub. No.: US 2021/0147946 A1
(43) Pub. Date: May 20, 2021

(54) SYSTEMS AND ASSAYS FOR ASSESSING MICROSATELLITE INSTABILITY

(71) Applicant: Life Technologies Corporation, Carlsbad, CA (US)

(72) Inventors: Charles Wendell Higdon, III, Pacifica, CA (US); Harrison Leong, San Francisco, CA (US); Charles Baudo, Oakland, CA (US); Carole Bornarth, Fremont, CA (US); Edgar Schreiber, Redwood City, CA (US)

(21) Appl. No.: 17/091,092

(22) Filed: Nov. 6, 2020

Related U.S. Application Data
(60) Provisional application No. 62/932,910, filed on Nov. 8, 2019.

(51) Int. Cl. C12Q 1/6886; C12Q 1/6844; G16B 30/10; G16B 40/10

(52) U.S. Cl. CPC C12Q 1/6886; C12Q 1/6844; G16B 30/10; C12Q 2600/16; C12Q 2600/106; C12Q 2600/112; G16B 40/10

(57) ABSTRACT

Systems, primers, kits, and methods for detecting microsatellite instability in a biological sample are described. Signal data is received from a capillary electrophoresis genetic analysis instrument, wherein the signal data is measured from fluorescence of fragments comprising nucleic acid sequences amplified from the biological sample via polymerase chain reaction (PCR). The nucleic acid sequences correspond to a plurality of different microsatellite loci and are obtained using a plurality of PCR primers configured to flank a plurality of microsatellite loci of a biological sample. When the PCR primers and the biological sample are combined and subjected to PCR amplification, fluorescently labeled DNA fragments are generated comprising the plurality of microsatellite loci. Fluorescent data obtained from the plurality of fluorescently labelled microsatellite loci are used to classify microsatellite instability of the biological sample.

### RELATED APPLICATIONS

This application which claims the right of priority under 35 U.S.C. § 119(e) to U.S. Provisional Appl. No. 62/932,910, filed Nov. 8, 2019, which is commonly owned this with application and which is hereby expressly incorporated by reference in its entirety as though fully set forth herein.

### SEQUENCE LISTING

The instant application contains a Sequence Listing which has been submitted electronically in ASCII format and is hereby incorporated by reference in its entirety. Said ASCII copy, created on Nov. 2, 2020, is named LT01514_SL.txt and is 7,735 bytes in size.

### BACKGROUND

This disclosure relates generally to DNA fragment analysis.

A causal factor in cancer is thought to be the breakdown of biomolecular machinery to repair DNA. During cell replication, DNA repair mechanisms are critical to the integrity of the replicate cells. When these mechanisms break down, mistakes can accumulate in the DNA carried by the resulting cells. There are cancer fighting drugs that take advantage of this breakdown to identify and destroy tumors. The drugs are most effective when tumors exhibit a high mutation rate which, in turn, is associated with a high degree of malfunction of the DNA repair biomolecular machinery. One way of detecting the circumstances in which the drugs would be most effective is to examine the degree to which DNA deviates from normal at loci where the DNA consists of many repeated subsequences. These subsequences are referred to as micro-satellites.

Microsatellites, also known as short tandem repeats (STRs), are polymorphic DNA loci consisting of short nucleotide sequences, usually 1-6 base repeats. These motifs comprise approximately 3% of the human genome. During DNA replication, these sequences are susceptible to errors that can result in deletions and insertions. When deficiencies in the DNA MMR system are present, microsatellite replication errors accumulate in the genome. This phenomenon is commonly referred to as microsatellite instability (MSI). In a typical microsatellite analysis, microsatellite loci are amplified by polymerase chain reaction (PCR) using fluorescently labeled forward primers and unlabeled reverse primers. The PCR amplicons are separated by size using electrophoresis. Applications include linkage mapping; animal breeding; human, animal, and plant typing; pathogen sub-typing; genetic diversity; microsatellite instability; Loss of Heterozygosity (LOH); Inter-simple sequence repeat (ISSR); Multilocus Variant Analysis (MLVA); and companion diagnostics for cancer treatments.

### SUMMARY

The instant technology generally relates to methods, systems, compositions, and kits for detecting microsatellite instability (MSI) in a DNA sample. When the number of microsatellites at a given DNA locus differs substantially from normal, that microsatellite locus is considered to be microsatellite unstable (MSU). When numerous microsatellite loci exhibit instability, the DNA sample is considered to have high microsatellite instability, MSI high. When there are only a few exhibiting instability, the DNA sample is considered to be MSI low. When none exhibit instability, the DNA sample is considered to be microsatellite stable, MSS.

In an aspect, a method for detecting microsatellite instability (MSI) in a DNA sample is provided, the method including: a) co-amplifying a plurality of microsatellite loci of the DNA sample to produce amplified fragments comprising nucleic acid sequences (e.g., DNA fragments) from each locus; b) determining the size of the amplified fragments from each locus; and c) comparing the size of the amplified fragments from each locus to the size of corresponding amplified fragments from a control. In embodiments, the plurality of loci includes at least one locus selected from BAT25, BAT 26, BAT40, CAT25, NR21, NR22, NR24, NR27, ABI-20A, ABI-17, ABI-16, ABI-20B and ABI-19. In embodiments, a difference in size between one or more amplified fragments between the sample and the control indicates the presence of MSI at the corresponding locus in the DNA sample.

In another aspect, a method for analyzing a DNA sample to determine microsatellite instability (MSI) in the DNA sample is provided, the method including: a) co-amplifying a plurality of microsatellite loci of the DNA sample to produce amplified fragments comprising nucleic acid sequences (e.g., DNA fragments) from each locus; b) determining the size of the amplified fragments from each locus; c) comparing the size of the amplified fragments from each locus to the size of corresponding amplified fragments from a control, a difference in size between one or more amplified fragments between the sample and the control indicating the presence of MSI in the DNA sample; and d) assigning a degree of MSI to the DNA sample, thereby determining the MSI status of the DNA sample. In embodiments, the plurality of loci includes at least one locus selected from BAT25, BAT 26, BAT40, CAT25, NR21, NR22, NR24, NR27, ABI-20A, ABI-17, ABI-16, ABI-20B and ABI-19.

In another aspect, a method for diagnosing the presence of cancerous tissue in a biological sample is provided, the method including: a) co-amplifying a plurality of microsatellite loci of the DNA sample to produce amplified fragments comprising nucleic acid sequences (e.g., DNA fragments) from each locus; b) determining the size of the amplified fragments from each locus; and c) comparing the size of the amplified fragments from each locus to the size of corresponding amplified fragments from a control. In embodiments, the plurality of loci includes at least one locus selected from BAT25, BAT 26, BAT40, CAT25, NR21, NR22, NR24, NR27, ABI-20A, ABI-17, ABI-16, ABI-20B and ABI-19. In embodiments, a difference in size between one or more amplified fragments between the sample and the control indicates the presence of microsatellite instability (MSI) at the corresponding locus in the DNA sample, wherein the presence of MSI in the DNA sample indicates that the biological sample contains cancerous tissue.

In embodiments, the method further includes assigning a degree of MSI to the DNA sample. In embodiments, the DNA sample is assigned a high degree of MSI (microsatellite instability high) if more than about 30% of the loci in the DNA sample are determined to have MSI. In embodiments, the DNA sample is assigned a low degree of MSI ((microsatellite instability low) if less than about 30% but more than about 1% of the loci in the DNA sample are determined to have MSI. In embodiments, the DNA sample is assigned a stable degree (microsatellite stable) if none of the loci in the DNA sample are determined to have MSI.

In another aspect, a method for diagnosing cancer in a subject having or suspected of having cancer is provided, the method including: a) co-amplifying a plurality of microsatellite loci of the DNA sample to produce amplified fragments comprising nucleic acid sequences (e.g., DNA fragments) from each locus; b) determining the size of the amplified fragments from each locus; and c) comparing the size of the amplified fragments from each locus to the size of corresponding amplified fragments from a control, a difference in size between one or more amplified fragments between the sample and the control indicating the presence of microsatellite instability (MSI) at the corresponding locus in the DNA sample. In embodiments, the presence of MSI in the DNA sample indicates that the subject has cancer. In embodiments, the plurality of loci includes at least one locus selected from BAT25, BAT 26, BAT40, CAT25, NR21, NR22, NR24, NR27, ABI-20A, ABI-17, ABI-16, ABI-20B and ABI-19.

In another aspect, a method for treating cancer in a subject having or suspected of having cancer is provided, the method including: a) co-amplifying a plurality of microsatellite loci of the DNA sample to produce amplified fragments comprising nucleic acid sequences (e.g., DNA fragments) from each locus; b) determining the size of the amplified fragments from each locus; c) comparing the size of the amplified fragments from each locus to the size of corresponding amplified fragments from a control; and d) administering to the subject having cancer an anti-cancer agent. In embodiments, a difference in size between one or more amplified fragments between the samples indicates the presence of microsatellite instability (MSI) in the DNA sample. In embodiments, the presence of MSI in the DNA sample indicates that the subject has cancer. In embodiments, the plurality of loci includes at least one locus selected from BAT25, BAT 26, BAT40, CAT25, NR21, NR22, NR24, NR27, ABI-20A, ABI-17, ABI-16, ABI-20B and ABI-19.

In another aspect, a method for analyzing amplified fragments comprising nucleic acid sequences (e.g., DNA fragments) to determine microsatellite instability (MSI) is provided, the method including: a) providing amplified fragments comprising nucleic acid sequences (e.g., DNA fragments) amplified from a plurality of microsatellite loci in a DNA sample; b) determining the size of the amplified fragments; c) comparing the size of the amplified fragments of step b) to the size of corresponding amplified fragments from a paired normal DNA sample, a difference in size between one or more fragments between the samples indicating the presence of MSI in the DNA sample; and d) assigning a degree of MSI to the DNA sample, thereby determining the MSI status of the DNA sample. In embodiments, the amplified fragments include a modification. In embodiments, the modification includes a detectable marker. In embodiments, the plurality of loci includes at least one locus selected from BAT25, BAT26, BAT40, CAT25, NR21, NR22, NR24, NR27, ABI-20A, ABI-17, ABI-16, ABI-20B and ABI-19.

In embodiments, the method further includes co-amplifying one or more identification markers in step a). In embodiments, the one or more identification markers include PENTAD and/or TH01.

In embodiments, the plurality of loci include at least two, three, four, five, six, or seven loci selected from BAT25, BAT26, BAT40, CAT25, NR21, NR22, NR24, NR27, ABI-20A, ABI-17, ABI-16, ABI-20B and ABI-19. In embodiments, the plurality of loci include at least eight, nine, ten, eleven, or twelve loci selected from BAT25, BAT26, BAT40, CAT25, NR21, NR22, NR24, NR27, ABI-20A, ABI-17, ABI-16, ABI-20B and ABI-19. In embodiments, the plurality of loci includes each of the following loci: BAT25, BAT26, BAT40, CAT25, NR21, NR22, NR24, NR27, ABI-20A, ABI-17, ABI-16, ABI-20B and ABI-19.

In embodiments, the DNA sample is from tumor cells, cells suspected of being cancerous, or other biological material suspected of being cancerous. In embodiments, the control is a paired normal DNA sample, a DNA sample from a non-cancerous tissue, DNA from a blood sample, an average based on a normal (non-cancerous) population, or a median based on a normal (non-cancerous) population.

In embodiments, the microsatellite loci are co-amplified using one or more primers selected from SEQ ID NOS.: 1-26. In embodiments, the microsatellite loci are co-amplified using a primer pair including a first primer and a second primer, wherein polynucleotide sequences of the first primer and the second primer include one of the following pairs: the polynucleotide sequence of SEQ ID NO.: 1 and the polynucleotide sequence of SEQ ID NO.: 2; the polynucleotide sequence of SEQ ID NO.: 3 and the polynucleotide sequence of SEQ ID NO.: 4; the polynucleotide sequence of SEQ ID NO.: 5 and the polynucleotide sequence of SEQ ID NO.: 6; the polynucleotide sequence of SEQ ID NO.: 7 and the polynucleotide sequence of SEQ ID NO.: 8; the polynucleotide sequence of SEQ ID NO.: 9 and the polynucleotide sequence of SEQ ID NO.: 10; the polynucleotide sequence of SEQ ID NO.: 11 and the polynucleotide sequence of SEQ ID NO.: 12; the polynucleotide sequence of SEQ ID NO.: 13 and the polynucleotide sequence of SEQ ID NO.: 14; the polynucleotide sequence of SEQ ID NO.: 15 and the polynucleotide sequence of SEQ ID NO.: 16; the polynucleotide sequence of SEQ ID NO.: 17 and the polynucleotide sequence of SEQ ID NO.: 18; the polynucleotide sequence of SEQ ID NO.: 19 and the polynucleotide sequence of SEQ ID NO.: 20; the polynucleotide sequence of SEQ ID NO.: 21 and the polynucleotide sequence of SEQ ID NO.: 22; the polynucleotide sequence of SEQ ID NO.: 23 and the polynucleotide sequence of SEQ ID NO.: 24; the polynucleotide sequence of SEQ ID NO.: 25 and the polynucleotide sequence of SEQ ID NO.: 26.

In embodiments, the plurality of microsatellite loci is amplified using thermal cycling, and analyzed via fragment analysis, Sanger sequencing, ion semiconductor sequencing, or high-resolution melt curve analysis. In embodiments, the Sanger sequencing is capillary electrophoresis Sanger sequencing. In embodiments, the DNA sample and the paired normal DNA sample are from the same individual. In embodiments, the paired normal DNA sample is a control DNA from a non-cancerous tissue. In embodiments, the DNA sample and the paired normal DNA sample are from the same type of tissue. In embodiments, the cancer is colorectal cancer, gastric cancer, adrenocortical carcinoma, cervical cancer, mesothelioma, or endometrial cancer. In embodiments, each locus is amplified using a primer pair, and further wherein at least one primer of the primer pair includes a modification. In embodiments, the modification includes a detectable marker.

In another aspect, a primer set is provided, including one or more primer pairs, each primer pair including a first primer and a second primer, wherein polynucleotide sequences of the first primer and the second primer include one of the following pairs: the polynucleotide sequence of SEQ ID NO.: 1 and the polynucleotide sequence of SEQ ID NO.: 2; the polynucleotide sequence of SEQ ID NO.: 3 and the polynucleotide sequence of SEQ ID NO.: 4; the polynucleotide sequence of SEQ ID NO.: 5 and the polynucleotide sequence of SEQ ID NO.: 6; the polynucleotide sequence of SEQ ID NO.: 7 and the polynucleotide sequence of SEQ ID NO.: 8; the polynucleotide sequence of SEQ ID NO.: 9 and the polynucleotide sequence of SEQ ID NO.: 10; the polynucleotide sequence of SEQ ID NO.: 11 and the polynucleotide sequence of SEQ ID NO.: 12; the polynucleotide sequence of SEQ ID NO.: 13 and the polynucleotide sequence of SEQ ID NO.: 14; the polynucleotide sequence of SEQ ID NO.: 15 and the polynucleotide sequence of SEQ ID NO.: 16; the polynucleotide sequence of SEQ ID NO.: 17 and the polynucleotide sequence of SEQ ID NO.: 18; the polynucleotide sequence of SEQ ID NO.: 19 and the polynucleotide sequence of SEQ ID NO.: 20; the polynucleotide sequence of SEQ ID NO.: 21 and the polynucleotide sequence of SEQ ID NO.: 22; the polynucleotide sequence of SEQ ID NO.: 23 and the polynucleotide sequence of SEQ ID NO.: 24; the polynucleotide sequence of SEQ ID NO.: 25 and the polynucleotide sequence of SEQ ID NO.: 26. In embodiments, at least one primer includes a modification. In embodiments, the modification includes a detectable label.

In another aspect, a composition is provided including a primer set of any one of the previously described primer sets, including embodiments. In embodiments, the primer set includes at least 2, at least 3, at least 4, at least 5, at least 6, at least 7, at least 8, at least 9, at least 10, at least 11, or at least 12 primer pairs. In embodiments, the composition includes 13 primer pairs including the polynucleotide sequences of SEQ ID NOs.: 1-26. In embodiments, at least one primer is modified. In embodiments, the modification includes a detectable label. In embodiments, the composition further includes a polymerase. In embodiments, the composition further includes a plurality of deoxyribonucleotide triphosphates. In embodiments, the composition further includes a DNA sample. In embodiments, the composition further includes one or more salts.

In another aspect, a system is provided, including the composition as previously described, including embodiments, and a first device configured to perform DNA amplification. In embodiments, the first device is configured to perform Sanger sequencing, ion semiconductor sequencing, capillary electrophoresis, or high-resolution melt analysis. In embodiments, the system further includes a second device configured to compare and/or analyze nucleic acid fragments resulting from amplification of DNA with the primers.

In another aspect, a kit including a buffer and at least one primer pair for amplification of a microsatellite locus is provided, the primer pair including a first primer and a second primer. In embodiments, the microsatellite locus includes BAT25, BAT26, BAT40, CAT25, NR21, NR22, NR24, NR27, ABI-20A, ABI-17, ABI-16, ABI-20B and/or ABI-19.

In embodiments, the at least one primer pair includes at least 2, at least 3, at least 4, at least 5, at least 6, at least 7, at least 8, at least 9, at least 10, at least 11, or at least 12, or at least 13 primer pairs. In embodiments, the at least one primer pair includes at least one primer having the polynucleotide sequence of any one of SEQ ID NOs.: 1-26. In embodiments, the at least one primer pair includes 13 primer pairs, the primers having the polynucleotide sequence of each of SEQ ID NOs.: 1-26. In embodiments, the kit further includes a primer pair for amplification of an identification marker. In embodiments, the identification marker includes PENTAD and/or TH01.

In embodiments, polynucleotide sequences of the first primer and the second primer include one of the following pairs: the polynucleotide sequence of SEQ ID NO.: 1 and the polynucleotide sequence of SEQ ID NO.: 2; the polynucleotide sequence of SEQ ID NO.: 3 and the polynucleotide sequence of SEQ ID NO.: 4; the polynucleotide sequence of SEQ ID NO.: 5 and the polynucleotide sequence of SEQ ID NO.: 6; the polynucleotide sequence of SEQ ID NO.: 7 and the polynucleotide sequence of SEQ ID NO.: 8; the polynucleotide sequence of SEQ ID NO.: 9 and the polynucleotide sequence of SEQ ID NO.: 10; the polynucleotide sequence of SEQ ID NO.: 11 and the polynucleotide sequence of SEQ ID NO.: 12; the polynucleotide sequence of SEQ ID NO.: 13 and the polynucleotide sequence of SEQ ID NO.: 14; the polynucleotide sequence of SEQ ID NO.: 15 and the polynucleotide sequence of SEQ ID NO.: 16; the polynucleotide sequence of SEQ ID NO.: 17 and the polynucleotide sequence of SEQ ID NO.: 18; the polynucleotide sequence of SEQ ID NO.: 19 and the polynucleotide sequence of SEQ ID NO.: 20; the polynucleotide sequence of SEQ ID NO.: 21 and the polynucleotide sequence of SEQ ID NO.: 22; the polynucleotide sequence of SEQ ID NO.: 23 and the polynucleotide sequence of SEQ ID NO.: 24; the polynucleotide sequence of SEQ ID NO.: 25 and the polynucleotide sequence of SEQ ID NO.: 26.

In embodiments, all primers of the primer pair(s) are present in the same container. In embodiments, the kit includes primers including the polynucleotide sequence of each of SEQ ID NO.: 1-26. In embodiments, the kit further includes a polymerase and/or a plurality of deoxynucleotide triphosphates. In embodiments, the buffer is a PCR buffer. In embodiments, at least one primer from the primer set includes a modification. In embodiments, the modification is a detectable label. In embodiments, the kit further includes a computer program for identification of microsatellite instability in a biological sample.

Embodiments of the invention used to detect microsatellite instability in a biological sample are disclosed. Signal data is received from a capillary electrophoresis genetic analysis instrument, wherein the signal data is measured from fluorescence of fragments comprising nucleic acid sequences amplified from the biological sample via polymerase chain reaction. The nucleic acid sequences correspond to a plurality of different microsatellite loci. Different loci can exhibit different signal characteristics. At a particular locus, a hierarchy of analysis methods can be applied that may also be peculiar to the characteristics of the signal data at that locus. For example, a three-level hierarchy could be described as follows: A first processing algorithm is implemented to obtain a first determination, based on the signal data, regarding instability of one or more first microsatellite loci of the plurality of different microsatellite loci. A second processing algorithm is implemented to obtain a second determination, based on the signal data, regarding instability of one or more second microsatellite loci of the plurality of different microsatellite loci. A third processing algorithm is then implemented to measure microsatellite instability of the biological sample based on at least the first determination and the second determination.

Embodiments of the invention describe a collection of ways to analyze the CE data to determine whether a given DNA locus is abnormal and to determine whether the overall genetic profile, combining results from all loci, can be considered MSI high, MSI low, or MSS. The methods described herein provide a means to automatically make the calls. The methods described can also be used to assign a confidence metric to the calls, for example, by reporting the proximity of calculated results to decision thresholds, which, in turn, can be used to focus human review efforts on those cases where the automated MSI assessment is less confident.

Embodiments of the present invention disclosed herein describe a heterogeneous approach to the analysis ranging from simple thresholds up to utilizing deep learning technologies. The reason for this is that assigning the overall genetic profile to MSI high, MSI low or MSI stable can involve one locus of microsatellites in the DNA up to many loci of microsatellites in the DNA. The complexity of analysis algorithms depends on the nature of DNA replication patterns at the loci chosen. Different loci might be chosen for different cancers since some may be more sensitive to a given cancer type compared to other cancer types and/or, in combination with other loci, may yield a more sensitive and/or specific test for MSI status, and/or the DNA may be more reliably amplified.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a system in accordance with an embodiment of the present invention;

FIG. 2 illustrates an exemplary capillary electrophoresis process used in some embodiments of the present invention;

FIG. 3 illustrates an exemplary genetic analyzer instrument used in some embodiments of the present invention;

FIG. 4 illustrates an exemplary all-in-one cartridge used in the exemplary genetic analyzer instrument of FIG. 3;

FIG. 5 illustrates four exemplary screenshots of user interface displays used in some embodiments of the present invention;

FIG. 6 illustrates a flow diagram depicting a cloud integration process of the exemplary genetic analyzer instrument of FIG. 3;

FIG. 7 illustrates a flow diagram of a method according to some embodiments of the present invention;

FIG. 8 illustrates a flow diagram of alternate methods according to some embodiments of the present invention;

FIG. 9 illustrates a block diagram of a distributed computer system that can implement one or more aspects of an embodiment of the invention; and

FIG. 10 illustrates a block diagram of an electronic device that can implement one or more aspects of an embodiment of the invention.

FIG. 11 shows a representative electropherogram of the 15-plex assay on DNA samples from colorectal carcinoma (left) or normal tissue (right), with the markers being spaced across the channels. Top row shows results from amplification of BAT25, NR24 and NR21 loci. Second row shows results from amplification of TH01, BAT40, and CAT25 loci. Third row shows results from amplification of NR22, NR27, ABI19, and ABI20B loci. Fourth row shows results from amplification of PentaD locus. Bottom row shows results from amplification of ABI17, ABI16, BAT26, and ABI20A loci. Loci are indicated by the gray bars over each trace, with divisions between each locus' trace indicated by red triangles on the X axis.

FIG. 12A is a table comparing the results of an MSI assay as described herein with a competitor (ProMega) MSI assay when run on endometrial carcinoma samples.

FIG. 12B shows trace profiles showing the clear shifts across the different types of markers: 20 bp (ABI-20A & ABI-20B) vs 40 bp (BAT-40) homopolymers.

FIG. 13 shows synthetic constructs revealing detection complexities.

FIG. 14 is a graph of tumor-only analysis with >98% specificity and >90% sensitivity at 5 bpd.

FIG. 15 is a graph of tumor-normal analysis with >95% specificity and sensitivity at ≥3 bdp.

FIG. 16 is an ABI MSI software illustration of automatic calls and reports comparing normal versus tumor colon samples at BAT-25, NR-24, and NR-21 (top) and all 15 loci (bottom).

While the invention is described with reference to the above drawings, the drawings are intended to be illustrative, and other embodiments are consistent with the spirit, and within the scope, of the invention.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “amplified DNA fragments” or “amplified fragments comprising nucleic acid sequences” refers to polynucleotide sequences that were produced by an in vitro amplification method, for example Polymerase Chain Reaction (PCR), isothermal amplification, strand displacement amplification, or any other DNA amplification method.

The term “microsatellite locus” or “microsatellite loci” refers to loci in the genome of an organism that contain a microsatellite. A “microsatellite” is a tract of repetitive DNA in which certain DNA motifs (generally from one to six or more base pairs) are repeated, typically 5-50 times. Microsatellites occur at thousands of locations within an organism's genome.

As used herein, the term “identification marker” refers to a locus that can be used to identify a DNA sample. Identification markers can be any locus that has allelic variability in a population, such that the allelic variability can be differentiated by the amplification used. Generally, such identification markers are short tandem repeat (STR) polymorphisms, and these polymorphisms can be differentiated based on length of the STR. For example, an identification marker may be any locus used in forensic DNA profiling, parentage determination, or similar methods. Such identification markers include, without limitation, Amelogenin, CSF1PO, D135317, D165539, D18551, D21S11, D3S1358, D5S818, D7S820, D8S1179, FGA, PentaD, PentaE, TH01, TPDX, and vWA. In embodiments, the identification marker is a single nucleotide polymorphism (SNP). In embodiments, the length of fragments comprising nucleic acid sequences amplified from the identification marker does not change or shows a lesser degree of change in cancers exhibiting MSI.

### Detection of MSI

The evaluation of MSI is increasingly being used by clinical researchers for multiple purposes, including: 1) To assess the effectiveness of oncology immunotherapy treatment options and; 2) To inform the diagnosis of a type of neoplastic inherited syndrome, termed Lynch Syndrome (Vaksman and Garner, 2015). Users have indicated that current market solutions are insufficient for testing in multiple cancer types, that analysis is slow, and data reporting is cumbersome. The proposed Microsatellite Instability assay aims to resolve these issues by developing a product that is sensitive across multiple cancer types and simplifies data analysis through automated genotyping.

MSI status is used as a predictive biomarker for cancer immunotherapy. Microsatellite instability, due to inherited germline mutations of mismatch repair genes or epigenetic inactivation of these genes, is found in many cancer types at varying frequencies. The tumors with the highest rates of MSI include uterine corpus endometrial carcinoma, colorectal adenocarcinoma, and stomach adenocarcinoma (Cortes-Ciriano et al, 2017).

Lynch syndrome is the most common inherited colorectal cancer (CRC) susceptibility syndrome. It accounts for approximately 3-5% of newly diagnosed causes of CRCs and 2-3% of endometrial cancers. The American Society of Clinical Oncology recommends that tumor testing for Lynch syndrome be performed in all people diagnosed with colorectal cancer. Recent guidelines recommend tumor testing for all endometrial cancers as well. Screening tests can be performed on tumor tissue to determine if Lynch syndrome is likely. One way to test is analyzing MSI. The result of MSI testing can indicate whether more specific genetic testing should be considered. Recent results indicate 16% of all cancers that are determined to be MSI-H (MSI-high) are associated with Lynch Syndrome, warranting more widespread testing for MSI, irrespective of tumor type or family history (Latham et al, 2019).

Fragment analysis has frequently been used to assess MSI status. Fragment analysis applications are those in which fluorescent fragments of DNA are produced by PCR using dye-labelled primers designed for a specific interrogation task. These fragments are then separated using capillary electrophoresis and sized by comparison to a size standard. MSI analysis via fragment analysis follows this same paradigm; PCR amplification of the microsatellite loci of interest using fluorescently labeled primers. The labeled PCR products are then analyzed by capillary electrophoresis (CE) or electrophoresis to separate the alleles by size.

When the proportion of DNA with microsatellites that differ from the normal molecules is low, it can be very hard to detect that abnormal DNA molecules are present. More accurate ways are needed to analyze the CE data to resolve the uncertainty enough to reliably distinguish between MSI high and MSI stable at a given DNA locus and to determine whether the overall genetic profile can be considered MSI high or MSI low.

There are possible alternatives to using CE fragment analysis. In a simple example, sequencing technologies can be used to sequence the DNA loci of interest and, through sequence analysis (e.g., counting the number of microsatellites in the sequence), assign MSI status. However, using sequencing technologies or similar approaches other than CE fragment analysis may be disadvantageous. For example, DNA sequence analysis has a limited ability to multiplex data. In addition, the process of DNA sequence analysis takes longer, and the analysis may be more error prone.

In an aspect, a method for detecting microsatellite instability (MSI) in a DNA sample is provided. In another aspect, a method for analyzing a DNA sample to determine microsatellite instability (MSI) in the DNA sample is provided. In another aspect, a method for diagnosing the presence of cancerous tissue in a biological sample is provided. In another aspect, a method for treating cancer in a subject having or suspected of having cancer is provided. In another aspect, a method for diagnosing cancer in a subject having or suspected of having cancer is provided

In embodiments, the method includes: a) co-amplifying a plurality of micro satellite loci of the DNA sample to produce amplified fragments comprising nucleic acid sequences from each locus; b) determining the size of the amplified fragments from each locus; and c) comparing the size of the amplified fragments from each locus to the size of corresponding amplified fragments from a control. In embodiments, the plurality of loci includes at least one locus selected from BAT25, BAT 26, BAT40, CAT25, NR21, NR22, NR24, NR27, ABI-20A, ABI-17, ABI-16, ABI-20B and ABI-19. In embodiments, a difference in size between one or more amplified fragments between the sample and the control indicates the presence of MSI at the corresponding locus in the DNA sample or biological sample.

In another aspect, a method for analyzing amplified fragments comprising nucleic acid sequences to determine microsatellite instability (MSI) is provided, the method including: a) providing amplified fragments amplified from a plurality of microsatellite loci in a DNA sample or biological sample; b) determining the size of the amplified fragments; c) comparing the size of the amplified fragments of step b) to the size of corresponding amplified fragments from a paired normal DNA sample or paired normal biological sample, a difference in size between one or more amplified fragments between the samples indicating the presence of MSI in the DNA sample; and d) assigning a degree of MSI to the DNA sample, thereby determining the MSI status of the DNA sample.

In embodiments, a difference in size between one or more amplified fragments between the sample and the control indicates the presence of microsatellite instability (MSI) at the corresponding locus in the DNA sample. In embodiments, the presence of MSI in the DNA sample indicates that the biological sample from which the DNA sample was derived contains cancerous tissue.

In embodiments, the method further includes assigning a degree of MSI to the DNA sample. In embodiments, the DNA sample is assigned a high degree of MSI (microsatellite instability high) if more than about 30% of the loci in the DNA sample are determined to have MSI. In embodiments, the DNA sample is assigned a low degree of MSI (microsatellite instability low) if less than about 30% but more than about 1% of the loci in the DNA sample are determined to have MSI. In embodiments, the DNA sample is assigned a stable degree (microsatellite stable) if none of the loci in the DNA sample are determined to have MSI. For example, when 13 loci are co-amplified, a DNA sample where at least 4 (or at least 3) loci are determined to have MSI is assigned a high degree of MSI (i.e., is MSI-high); a DNA sample where at least 1 but less than 4 (or less than 3) loci are determined to have MSI is assigned a low degree of MSI (i.e., is MSI-low); and a DNA sample where 0 loci are determined to have MSI is assigned a stable degree of MSI (i.e., is MSI-stable).

In embodiments, the presence of MSI in the DNA sample indicates that the subject has cancer. In embodiments, the method includes administering to the subject having cancer an anti-cancer agent.

In embodiments, a primer used to amplify a locus includes a modification. In embodiments, each locus is amplified using a primer pair, and at least one primer of the primer pair includes a modification. In embodiments the modification may be migration modifiers such as poly ethylene glycol (PEG), locked nucleic acids (LNA), 3′-minor groove binders or the addition of non-specific nucleic acids to one end of the primer sequence. In embodiments, the amplified fragments include a modification. In embodiments, the modification includes a detectable marker. Detectable markers include, without limitation, fluorescent markers/dyes and radioactive markers. Fluorescent markers/dyes are well known in the art, and include, without limitation, fluorescein, FAM (carboxyfluorescein) (e.g., 5-FAM, 6-FAM), TET, VIC (2′-chloro-7′phenyl-1,4-dichloro-6-carboxy-fluorescein), HEX, NED, PET, JOE, TET, Cal Fluor Orange 560, TAMRA, QUASAR® 570, Cal Fluor Red, ROX™, Texas Red®, Quasar 670, LC Red 640, LC Red 705, SID, TAZ, YY, rhodamine (or derivatives), coumarin (or derivatives), and cyanine (or derivatives, e.g. Cy2, Cy3, Cy3B, Cy3.5, Cy5, Cy5.5, Cy7), or derivatives of any of these molecules.

In embodiments, the method further includes co-amplifying one or more identification markers in step a). In embodiments, the one or more identification markers include Amelogenin, CSF1PO, D135317, D165539, D18551, D21511, D3S1358, D5S818, D7S820, D8S1179, FGA, PentaD, PentaE, TH01, TPDX, and/or vWA. In embodiments, the one or more identification markers include PENTAD. In embodiments, the one or more identification markers include TH01. In embodiments, the one or more identification markers include PENTAD and TH01.

In embodiments, the term “comparing the size of” refers to comparison of the size or size distribution of nucleic acid fragments. Comparison can be performed by any method, such as gel electrophoresis, capillary electrophoresis, DNA sequencing, microscopic visualization (e.g., adsorption grid electron microscopic visualization or Kleinschmidt Electron Microscopic visualization), and the like. Comparison may be performed, for example, by visualization of the fragment size, and/or by a computer-implemented program.

In embodiments, the plurality of loci include at least 2 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 3 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 4 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 5 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 6 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 7 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 8 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 9 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 10 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 11 loci selected from BAT25, BAT26, BAT40, CAT25, NR21, NR22, NR24, NR27, ABI-20A, ABI-17, ABI-16, ABI-20B and ABI-19. In embodiments, the plurality of loci include at least 12 loci selected from BAT25, BAT26, BAT40, CAT25, NR21, NR22, NR24, NR27, ABI-20A, ABI-17, ABI-16, ABI-20B and ABI-19. In embodiments, the plurality of loci includes BAT25, BAT26, BAT40, CAT25, NR21, NR22, NR24, NR27, ABI-20A, ABI-17, ABI-16, ABI-20B and ABI-19.

In embodiments, the plurality of loci includes BAT25. In embodiments, the plurality of loci includes BAT26. In embodiments, the plurality of loci includes BAT40. In embodiments, the plurality of loci includes CAT25. In embodiments, the plurality of loci includes NR21. In embodiments, the plurality of loci includes NR22. In embodiments, the plurality of loci includes NR24. In embodiments, the plurality of loci includes NR27. In embodiments, the plurality of loci includes ABI-20A. In embodiments, the plurality of loci includes ABI-17. In embodiments, the plurality of loci includes ABI-16. In embodiments, the plurality of loci includes ABI-20B. In embodiments, the plurality of loci includes ABI-19.

In embodiments, the DNA sample is from a biological sample. In embodiments, the biological sample includes tumor cells, cells suspected of being cancerous, or other biological material suspected of being cancerous or from a cancer cell.

In embodiments, the control is a paired normal DNA sample, a DNA sample from a non-cancerous tissue, DNA from a blood sample, an average based on a normal population, or a median based on a normal population. In embodiments, the term “normal” refers to a DNA sample from non-cancerous tissue. In embodiments, the term “normal” refers to a DNA sample from non-cancerous tissue adjacent to cancerous tissue.

In embodiments, the microsatellite loci are co-amplified using one or more primers selected from SEQ ID NOS.: 1-26. In embodiments, the microsatellite loci are co-amplified using a primer containing the nucleotide sequence of SEQ ID NO.: 1. In embodiments, the microsatellite loci are co-amplified using a primer containing the nucleotide sequence of SEQ ID NO.: 2. In embodiments, the microsatellite loci are co-amplified using a primer containing the nucleotide sequence of SEQ ID NO.: 3. In embodiments, the microsatellite loci are co-amplified using a primer containing the nucleotide sequence of SEQ ID NO.: 4. In embodiments, the microsatellite loci are co-amplified using a primer containing the nucleotide sequence of SEQ ID NO.: 5. In embodiments, the microsatellite loci are co-amplified using a primer containing the nucleotide sequence of SEQ ID NO.: 6. In embodiments, the microsatellite loci are co-amplified using a primer containing the nucleotide sequence of SEQ ID NO.: 7. In embodiments, the microsatellite loci are co-amplified using a primer containing the nucleotide sequence of SEQ ID NO.: 8. In embodiments, the microsatellite loci are co-amplified using a primer containing the nucleotide sequence of SEQ ID NO.: 9. In embodiments, the microsatellite loci are co-amplified using a primer containing the nucleotide sequence of SEQ ID NO.: 10. In embodiments, the microsatellite loci are co-amplified using a primer containing the nucleotide sequence of SEQ ID NO.: 11. In embodiments, the microsatellite loci are co-amplified using a primer containing the nucleotide sequence of SEQ ID NO.: 12. In embodiments, the microsatellite loci are co-amplified using a primer containing the nucleotide sequence of SEQ ID NO.: 13. In embodiments, the microsatellite loci are co-amplified using a primer containing the nucleotide sequence of SEQ ID NO.: 14. In embodiments, the microsatellite loci are co-amplified using a primer containing the nucleotide sequence of SEQ ID NO.: 15. In embodiments, the microsatellite loci are co-amplified using a primer containing the nucleotide sequence of SEQ ID NO.: 16. In embodiments, the microsatellite loci are co-amplified using a primer containing the nucleotide sequence of SEQ ID NO.: 17. In embodiments, the microsatellite loci are co-amplified using a primer containing the nucleotide sequence of SEQ ID NO.: 18. In embodiments, the microsatellite loci are co-amplified using a primer containing the nucleotide sequence of SEQ ID NO.: 19. In embodiments, the microsatellite loci are co-amplified using a primer containing the nucleotide sequence of SEQ ID NO.: 20. In embodiments, the microsatellite loci are co-amplified using a primer containing the nucleotide sequence of SEQ ID NO.: 21. In embodiments, the microsatellite loci are co-amplified using a primer containing the nucleotide sequence of SEQ ID NO.: 22. In embodiments, the microsatellite loci are co-amplified using a primer containing the nucleotide sequence of SEQ ID NO.: 23. In embodiments, the microsatellite loci are co-amplified using a primer containing the nucleotide sequence of SEQ ID NO.: 24. In embodiments, the microsatellite loci are co-amplified using a primer containing the nucleotide sequence of SEQ ID NO.: 25. In embodiments, the microsatellite loci are co-amplified using a primer containing the nucleotide sequence of SEQ ID NO.: 26.

In embodiments, the microsatellite loci are co-amplified using a primer pair including a first primer and a second primer. In embodiments, the polynucleotide sequences of the first primer and the second primer include one of the following pairs (respectively): the polynucleotide sequence of SEQ ID NO.: 1 and the polynucleotide sequence of SEQ ID NO.: 2; the polynucleotide sequence of SEQ ID NO.: 3 and the polynucleotide sequence of SEQ ID NO.: 4; the polynucleotide sequence of SEQ ID NO.: 5 and the polynucleotide sequence of SEQ ID NO.: 6; the polynucleotide sequence of SEQ ID NO.: 7 and the polynucleotide sequence of SEQ ID NO.: 8; the polynucleotide sequence of SEQ ID NO.: 9 and the polynucleotide sequence of SEQ ID NO.: 10; the polynucleotide sequence of SEQ ID NO.: 11 and the polynucleotide sequence of SEQ ID NO.: 12; the polynucleotide sequence of SEQ ID NO.: 13 and the polynucleotide sequence of SEQ ID NO.: 14; the polynucleotide sequence of SEQ ID NO.: 15 and the polynucleotide sequence of SEQ ID NO.: 16; the polynucleotide sequence of SEQ ID NO.: 17 and the polynucleotide sequence of SEQ ID NO.: 18; the polynucleotide sequence of SEQ ID NO.: 19 and the polynucleotide sequence of SEQ ID NO.: 20; the polynucleotide sequence of SEQ ID NO.: 21 and the polynucleotide sequence of SEQ ID NO.: 22; the polynucleotide sequence of SEQ ID NO.: 23 and the polynucleotide sequence of SEQ ID NO.: 24; the polynucleotide sequence of SEQ ID NO.: 25 and the polynucleotide sequence of SEQ ID NO.: 26.

In embodiments, the identification marker(s) is co-amplified using one or more primers selected from SEQ ID NOS.: 27-31. In embodiments, the identification marker(s) is co-amplified using a primer containing the nucleotide sequence of SEQ ID NO.: 27. In embodiments, the identification marker(s) is co-amplified using a primer containing the nucleotide sequence of SEQ ID NO.: 28. In embodiments, the identification marker(s) is co-amplified using a primer containing the nucleotide sequence of SEQ ID NO.: 29. In embodiments, the identification marker(s) is co-amplified using a primer containing the nucleotide sequence of SEQ ID NO.: 30. In embodiments, the identification marker(s) is co-amplified using a primer containing the nucleotide sequence of SEQ ID NO.: 31.

In embodiments, the plurality of microsatellite loci is amplified using thermal cycling. In embodiments, the resulting nucleic acid fragments are analyzed via fragment analysis, Sanger sequencing, ion semiconductor sequencing, or high-resolution melt curve analysis. In embodiments, the Sanger sequencing is capillary electrophoresis Sanger sequencing.

In embodiments, the DNA sample and the paired normal DNA sample are from the same individual. In embodiments, the paired normal DNA sample is a control DNA from a non-cancerous tissue. In embodiments, the DNA sample and the paired normal DNA sample are from the same type of tissue.

The cancer can be any type of cancer. In particular, the cancer can be any cancer that is associated with MSI. In embodiments, the cancer is colorectal cancer, gastric cancer, adrenocortical carcinoma, cervical cancer, mesothelioma, or endometrial cancer.

At a given microsatellite locus, capillary electrophoresis (CE) can be used to measure the number of microsatellites by using fragment analysis. Automated CE uses fluorescent dyes and separates with higher resolution and higher accuracy than other methods such as agarose or polyacrylamide gel electrophoresis.

To run fragment analysis on a CE system, probes and primers can be designed to flank a region of interest. This can be done by attaching fluorescent dyes to primers or probes used with the polymerase chain reaction (PCR) to amplify a DNA locus of interest before the electrophoresis and submitting the amplicons to CE. There is also a sizing standard, a collection of fragments of known sizes labelled with a color that is different than the colors of the test fragments. The labelled PCR products and the sizing standard are then electrokinetically injected into the capillaries. During electrophoresis, the negatively charged DNA fragments moves from the cathode, through the polymer-filled capillary towards the positively charged anode when high voltage is applied between the electrodes.

DNA fragment analysis using CE can be multiplexed, meaning there are multiple fragments in a reaction well going through the same capillary. The smaller fragments usually run faster, and the bigger ones run slower. Shortly before reaching the positive electrode, the fluorescently labelled DNA fragments, separated by size, move through the path of a laser beam. The laser beam causes the dyes on the fragments to fluoresce at different emission wavelengths. A CCD camera detects the fluorescence, and the fluorescence intensities are digitalized, color-coded and displayed as peaks in the electropherogram. Longer fragments will occur later in the data relative to shorter fragments.

### Reagents and Kits

In another aspect, a primer set is provided. The primer set may include one or more primer pairs, each primer pair including a first primer and a second primer (e.g., forward and reverse primers). In embodiments, the polynucleotide sequences of the first primer and the second primer include one of the following pairs: the polynucleotide sequence of SEQ ID NO.: 1 and the polynucleotide sequence of SEQ ID NO.: 2; the polynucleotide sequence of SEQ ID NO.: 3 and the polynucleotide sequence of SEQ ID NO.: 4; the polynucleotide sequence of SEQ ID NO.: 5 and the polynucleotide sequence of SEQ ID NO.: 6; the polynucleotide sequence of SEQ ID NO.: 7 and the polynucleotide sequence of SEQ ID NO.: 8; the polynucleotide sequence of SEQ ID NO.: 9 and the polynucleotide sequence of SEQ ID NO.: 10; the polynucleotide sequence of SEQ ID NO.: 11 and the polynucleotide sequence of SEQ ID NO.: 12; the polynucleotide sequence of SEQ ID NO.: 13 and the polynucleotide sequence of SEQ ID NO.: 14; the polynucleotide sequence of SEQ ID NO.: 15 and the polynucleotide sequence of SEQ ID NO.: 16; the polynucleotide sequence of SEQ ID NO.: 17 and the polynucleotide sequence of SEQ ID NO.: 18; the polynucleotide sequence of SEQ ID NO.: 19 and the polynucleotide sequence of SEQ ID NO.: 20; the polynucleotide sequence of SEQ ID NO.: 21 and the polynucleotide sequence of SEQ ID NO.: 22; the polynucleotide sequence of SEQ ID NO.: 23 and the polynucleotide sequence of SEQ ID NO.: 24; the polynucleotide sequence of SEQ ID NO.: 25 and the polynucleotide sequence of SEQ ID NO.: 26.

In embodiments, at least one primer includes a modification. In embodiments, at least one primer in each primer pair comprises a modification. In embodiments, the modification includes a detectable label.

In another aspect, a composition is provided including a primer set of any one of the previously described primer sets, including embodiments. In embodiments, the primer set includes at least 2 primer pairs. In embodiments, the primer set includes at least 3 primer pairs. In embodiments, the primer set includes at least 4 primer pairs. In embodiments, the primer set includes at least 5 primer pairs. In embodiments, the primer set includes at least 6 primer pairs. In embodiments, the primer set includes at least 7 primer pairs. In embodiments, the primer set includes at least 8 primer pairs. In embodiments, the primer set includes at least 9 primer pairs. In embodiments, the primer set includes at least 10 primer pairs. In embodiments, the primer set includes at least 11 primer pairs. In embodiments, the primer set includes at least 12 primer pairs. In embodiments, the composition includes at least 13 primer pairs.

In embodiments, the primer pairs include the polynucleotide sequence of one or more of SEQ ID NOs.: 1-26. In embodiments, the primer pairs include the polynucleotide sequence of SEQ ID NO.: 1. In embodiments, the primer pairs include the polynucleotide sequence of SEQ ID NO.: 2. In embodiments, the primer pairs include the polynucleotide sequence of SEQ ID NO.: 3. In embodiments, the primer pairs include the polynucleotide sequence of SEQ ID NO.: 4. In embodiments, the primer pairs include the polynucleotide sequence of SEQ ID NO.: 5. In embodiments, the primer pairs include the polynucleotide sequence of SEQ ID NO.: 6. In embodiments, the primer pairs include the polynucleotide sequence of SEQ ID NO.: 7. In embodiments, the primer pairs include the polynucleotide sequence of SEQ ID NO.: 8. In embodiments, the primer pairs include the polynucleotide sequence of SEQ ID NO.: 9. In embodiments, the primer pairs include the polynucleotide sequence of SEQ ID NO.: 10. In embodiments, the primer pairs include the polynucleotide sequence of SEQ ID NO.: 11. In embodiments, the primer pairs include the polynucleotide sequence of SEQ ID NO.: 12. In embodiments, the primer pairs include the polynucleotide sequence of SEQ ID NO.: 13. In embodiments, the primer pairs include the polynucleotide sequence of SEQ ID NO.: 14. In embodiments, the primer pairs include the polynucleotide sequence of SEQ ID NO.: 15. In embodiments, the primer pairs include the polynucleotide sequence of SEQ ID NO.: 16. In embodiments, the primer pairs include the polynucleotide sequence of SEQ ID NO.: 17. In embodiments, the primer pairs include the polynucleotide sequence of SEQ ID NO.: 18. In embodiments, the primer pairs include the polynucleotide sequence of SEQ ID NO.: 19. In embodiments, the primer pairs include the polynucleotide sequence of SEQ ID NO.: 20. In embodiments, the primer pairs include the polynucleotide sequence of SEQ ID NO.: 21. In embodiments, the primer pairs include the polynucleotide sequence of SEQ ID NO.: 22. In embodiments, the primer pairs include the polynucleotide sequence of SEQ ID NO.: 23. In embodiments, the primer pairs include the polynucleotide sequence of SEQ ID NO.: 24. In embodiments, the primer pairs include the polynucleotide sequence of SEQ ID NO.: 25. In embodiments, the primer pairs include the polynucleotide sequence of SEQ ID NO.: 26.

In embodiments, the primer set further includes a primer pair for amplification of an identification marker. In embodiments, the identification marker includes PENTAD. and/or TH01. In embodiments, the identification marker includes TH01. In embodiments, the identification marker includes PENTAD and TH01. In embodiments, the primer pair for amplification of PENTAD comprises primers including the polynucleotide sequence of one or more of SEQ ID NO.: 27-29. In embodiments, the primer pair for amplification of TH01 comprises primers including the polynucleotide sequence of one or more of SEQ ID NO.: 30-31. In embodiments, the primers include a primer containing the nucleotide sequence of SEQ ID NO.: 27. In embodiments, the primers include a primer containing the nucleotide sequence of SEQ ID NO.: 28. In embodiments, the primers include a primer containing the nucleotide sequence of SEQ ID NO.: 29. In embodiments, the primers include a primer containing the nucleotide sequence of SEQ ID NO.: 30. In embodiments, the primers include using a primer containing the nucleotide sequence of SEQ ID NO.: 31.

In embodiments, at least one primer is modified. In embodiments, the modification includes a detectable label. In embodiments, the composition further includes a polymerase. In embodiments, the composition further includes a plurality of deoxyribonucleotide triphosphates. In embodiments, the composition further includes a DNA sample. In embodiments, the composition further includes one or more salts.

In another aspect, a kit including a buffer and a primer set including at least one primer pair for amplification of a microsatellite locus is provided, the primer pair including a first primer and a second primer. In embodiments, the microsatellite locus includes BAT25, BAT26, BAT40, CAT25, NR21, NR22, NR24, NR27, ABI-20A, ABI-17, ABI-16, ABI-20B and/or ABI-19.

In embodiments, the primer set includes at least 2 primer pairs. In embodiments, the primer set includes at least 3 primer pairs. In embodiments, the primer set includes at least 4 primer pairs. In embodiments, the primer set includes at least 5 primer pairs. In embodiments, the primer set includes at least 6 primer pairs. In embodiments, the primer set includes at least 7 primer pairs. In embodiments, the primer set includes at least 8 primer pairs. In embodiments, the primer set includes at least 9 primer pairs. In embodiments, the primer set includes at least 10 primer pairs. In embodiments, the primer set includes at least 11 primer pairs. In embodiments, the primer set includes at least 12 primer pairs. In embodiments, the primer set includes at least 13 primer pairs. In embodiments, the primer set includes at least 14 primer pairs. In embodiments, the primer set includes at least 14 primer pairs. In embodiments, the primer set includes at least one primer having the polynucleotide sequence of any one of SEQ ID NOs.: 1-26. In embodiments, the primer set includes 13 primer pairs, the primers having the polynucleotide sequence of each of SEQ ID NOs.: 1-26. In embodiments, the primer set is a primer set as described above.

In embodiments, the primer set further includes a primer pair for amplification of an identification marker. In embodiments, the identification marker includes PENTAD. In embodiments, the identification marker includes TH01. In embodiments, the identification marker includes PENTAD and TH01. In embodiments, the primer set includes primers having the polynucleotide sequence of any one or more of SEQ ID NOs.: 27-31.

In embodiments, polynucleotide sequences of the first primer and the second primer include one of the following pairs: the polynucleotide sequence of SEQ ID NO.: 1 and the polynucleotide sequence of SEQ ID NO.: 2; the polynucleotide sequence of SEQ ID NO.: 3 and the polynucleotide sequence of SEQ ID NO.: 4; the polynucleotide sequence of SEQ ID NO.: 5 and the polynucleotide sequence of SEQ ID NO.: 6; the polynucleotide sequence of SEQ ID NO.: 7 and the polynucleotide sequence of SEQ ID NO.: 8; the polynucleotide sequence of SEQ ID NO.: 9 and the polynucleotide sequence of SEQ ID NO.: 10; the polynucleotide sequence of SEQ ID NO.: 11 and the polynucleotide sequence of SEQ ID NO.: 12; the polynucleotide sequence of SEQ ID NO.: 13 and the polynucleotide sequence of SEQ ID NO.: 14; the polynucleotide sequence of SEQ ID NO.: 15 and the polynucleotide sequence of SEQ ID NO.: 16; the polynucleotide sequence of SEQ ID NO.: 17 and the polynucleotide sequence of SEQ ID NO.: 18; the polynucleotide sequence of SEQ ID NO.: 19 and the polynucleotide sequence of SEQ ID NO.: 20; the polynucleotide sequence of SEQ ID NO.: 21 and the polynucleotide sequence of SEQ ID NO.: 22; the polynucleotide sequence of SEQ ID NO.: 23 and the polynucleotide sequence of SEQ ID NO.: 24; the polynucleotide sequence of SEQ ID NO.: 25 and the polynucleotide sequence of SEQ ID NO.: 26.

In embodiments, one or more primers in the primer set are modified. In embodiments, at least one primer in each primer pair of the primer set is modified. In embodiments, the modification includes a detectable label.

In embodiments, all primers of the primer set are present in the same container. In embodiments, the kit further includes a polymerase. In embodiments, the kit further includes a plurality of deoxynucleotide triphosphates. In embodiments, the buffer is a PCR buffer. In embodiments, the kit further includes a computer program for identification of microsatellite instability in a biological sample.

### Systems and Analysis Methods

In another aspect, a system is provided, including a composition as previously described, including embodiments, and a first device configured to perform DNA amplification. In embodiments, the first device is configured to perform Sanger sequencing, ion semiconductor sequencing, capillary electrophoresis, or high-resolution melt analysis. In embodiments, the system further includes a second device configured to compare and/or analyze nucleic acid fragments resulting from amplification of DNA with the primers.

In another aspect, a system is provided, including a composition as previously described, including embodiments, and a device configured to compare and/or analyze nucleic acid fragments resulting from amplification of DNA with the primers.

Prior art solutions involving manual review of CE fragment analysis data to make MSI status calls tend to be rather time-consuming and an inefficient use of limited manual review time. Embodiments of the present invention discussed herein provide very wide coverage of reasonable methods to automatically make MSI status calls in many cases. The methods described can also be used to assign a confidence metric to the calls, for example, by reporting the proximity of calculated results to decision thresholds, which, in turn, can be used to focus human review efforts on those cases where the automated MSI assessment is less confident.

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

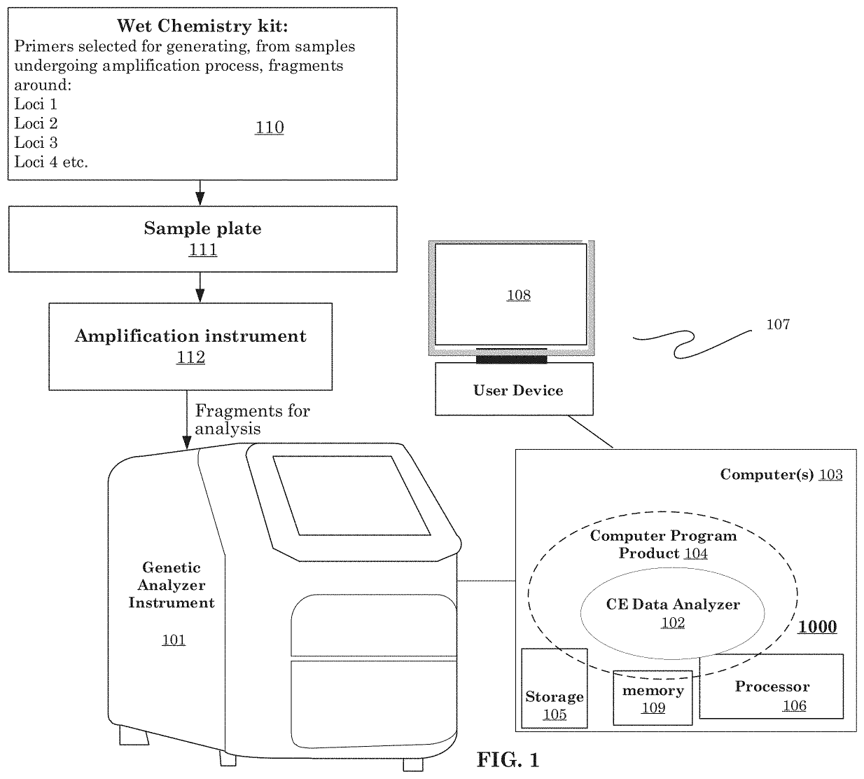

FIG. 1 illustrates system 1000 in accordance with an exemplary embodiment of the present invention. The DNA fragment analysis processes set forth in embodiments of the present invention start with extracting the DNA from a tissue sample. A plurality of microsatellite DNA loci of interest in one or more nucleic acid samples 111 under investigation may be amplified in a PCR reaction performed in amplification instrument 112 such as a thermal cycler. Exemplary thermal cycle instruments used in some embodiments of the present invention include the Applied Biosystems ProFlex PCR System, the SimpliAmp Thermal Cycler, and other thermal cycler systems manufactured by Thermo Fisher Scientific. The PCR reaction is performed using a wet chemistry kit 110 containing specially designed fluorescently labeled primers to flank the microsatellite loci of interest, such as those described herein. System 1000 comprises capillary electrophoresis based genetic analyzer instrument (e.g. a sequencing instrument) 101, one or more computers 103, and user device 108. Exemplary genetic analyzer instruments used in some embodiments of the present invention include the Applied Biosystems SeqStudio Genetic Analyzer by Thermo Fisher Scientific, Models 3500, 3720, and 3130 and similar capillary electrophoresis-based genetic analyzers manufactured by Thermo Fisher Scientific and others.

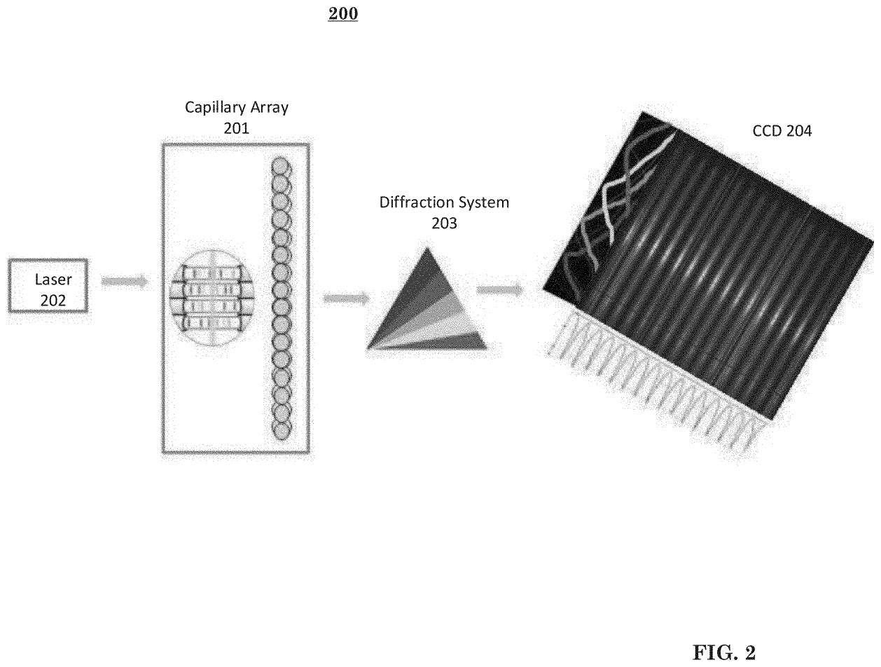

As shown in an exemplary process set forth in FIG. 2, capillary electrophoresis (CE) is a process 200 used to separate ionic fragments by size. In Thermo Fisher Scientific CE instrumentation used in some embodiments of the invention, an electrokinetic injection is used to inject DNA fragments from solution and into each capillary of a capillary array 201 comprising one or more capillaries. During capillary electrophoresis, the extension products of the PCR reaction (and any other negatively charged molecules such as salt or unincorporated primers and nucleotides) enter the capillary as a result of electrokinetic injection. A high voltage charge applied to the sample forces the negatively charged fragments into the capillaries. The extension products are separated by size based on their total charge. The electrophoretic mobility of the sample can be affected by the run conditions: the buffer type, concentration, and pH; the run temperature; the amount of voltage applied; and the type of polymer used.

Shortly before reaching the positive electrode, the fluorescently labeled DNA fragments, separated by size, move across the path of a laser beam 202. The laser beam causes the dyes attached to the fragments to fluoresce. The dye signals are separated by a diffraction system 203, and a CCD camera detects the fluorescence as shown in 204. Because each dye emits light at a different wavelength when excited by the laser, all colors, and therefore loci, can be detected and distinguished in one capillary injection. The fluorescence signal is converted into digital data, then the data is stored in a file format compatible with an analysis software application.

In general, the data coming out of the CE instrumentation is a series of fluorescent peaks instead of a single peak at the exact size of the amplicon expected for a given number of microsatellites. This is caused by nuances in the amplification of the DNA of interest; “stutter” of the biomolecular machinery involved can result in generating amplicons with a few more or a few less microsatellites in the amplicons than the true number of microsatellites. As a result of this “stutter”, there can be some uncertainty in determining the number of microsatellites and/or in determining whether the number of microsatellites differs from the number expected in normal, non-cancerous tissue.

Adding to the complexity of the signals received from the CE instrumentation, a single dye may be used with several different PCR primers that target different DNA loci. This is done because the instrumentation imposes limitations on the number of different dyes that can be used, and the number of DNA loci of interest may exceed the maximum number of dyes that can be used. If the amplicon sizes are sufficiently different between a group of loci for which the same dye is used on their respective PCR primers, the fluorescent peaks associated with each of the loci would be well separated in the data generated by the CE instrument.

Instructions for implementing the CE data analysis algorithms 102 shown in FIG. 1 reside in computer program product 104 which is stored in storage 105 and those instructions are executable by processor 106. When processor 106 is executing the instructions of computer program product 104, the instructions, or a portion thereof, are typically loaded into working memory 109 from which the instructions are readily accessed by processor 106. In the illustrated embodiment, computer program product 104 is stored in storage 105 or other non-transitory computer readable medium (which may include being distributed across media on different devices and different locations). In alternative embodiments, the storage medium is transitory.

In one embodiment, processor 106 in fact comprises multiple processors which may comprise additional working memories (additional processors and memories not individually illustrated) including a graphics processing unit (GPU) comprising at least thousands of arithmetic logic units supporting parallel computations on a large scale. GPUs are often utilized in deep learning applications because they can perform the relevant processing tasks more efficiently than can typical general-purpose processors (CPUs). Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application specific), field programmable gate arrays and the like, and combinations thereof. In some embodiments, however, a processor such as processor 106 may be implemented as one or more general purpose processors (preferably having multiple cores) without necessarily departing from the spirit and scope of the present invention.

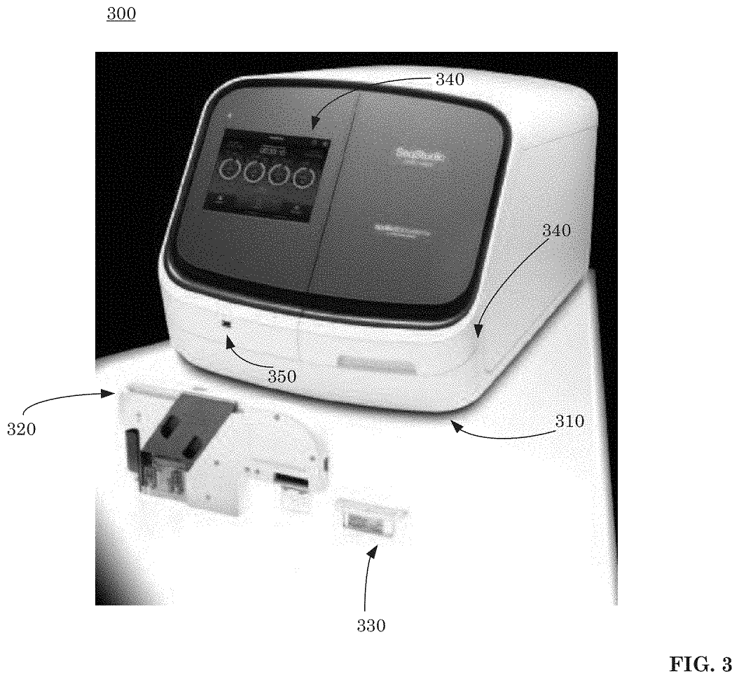

FIG. 3 shows an exemplary genetic analyzer instrument system 300 that may be used in the system of FIG. 1. In some embodiments of the present invention, exemplary genetic analyzer instrument system 300 comprises an Applied Biosystems™ SegStudio™ Genetic Analyzer instrument system, manufactured by ThermoFisher Scientific Inc., although other genetic analyzer instrument systems similarly capable of performing capillary electrophoresis may be used.

System 300 comprises genetic analyzer instrument 310, all-in-one cartridge 320, and cathode buffer 330. Built into genetic analyzer instrument 310 is touchscreen display 340 and USB port 350. Genetic analyzer system 300 used in some embodiments of the present invention allows multiple fragment analysis and/or sequencing runs on the same plate. Genetic analyzer system 300 is easy to use with integrated cartridge-based system 320 and allows researchers to access and monitor experimental runs as well as view data on the integrated touchscreen display 340, or remotely. The fully connected genetic analyzer, along with the simple cartridge design, can be easily shared by multiple researchers in a lab or facility.

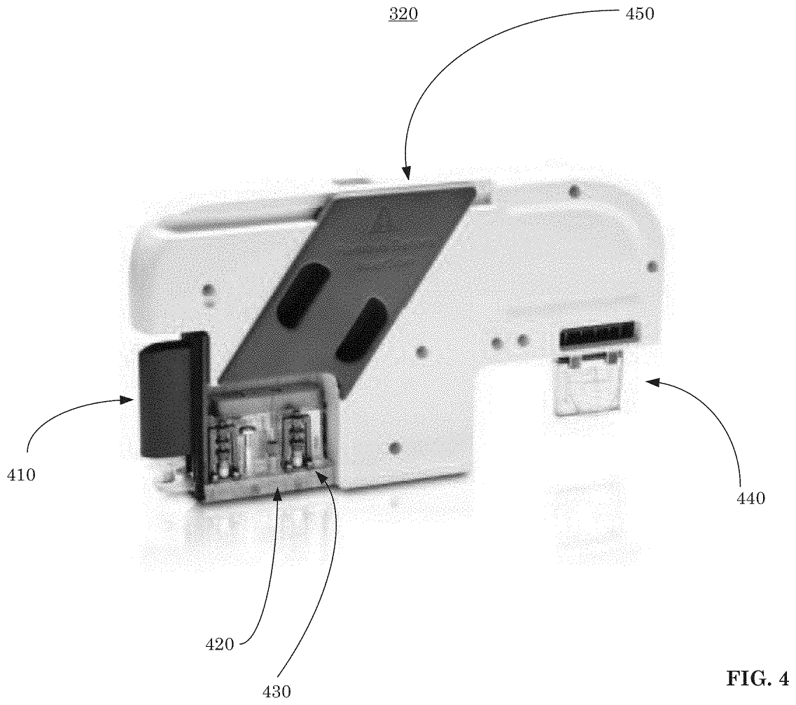

In some embodiments of the present invention, an easy-to-use functional core of the instrument includes a cartridge design that helps maximize efficiency and convenience. For example, the SeqStudio Genetic Analyzer mentioned above utilizes an all-in-one cartridge 320, shown in more detail in FIG. 4, that contains the capillary array 440, polymer reservoir 410, polymer delivery system 420, and anode buffer 430. A laser detection window may also be provided in some embodiments of cartridge 320. Cartridge 320 is removable and can be stored on the instrument for up to four months. In some embodiments of the present invention, each cartridge contains a new polymer unique to the SeqStudio system that allows Sanger sequencing and fragment analysis to be performed with no reconfiguration. In one embodiment of the present invention, the cartridge has four capillaries, and can process samples from either standard 96-well plates or 8-well strip tubes. In some embodiments of the present invention, the cartridge and cathode buffer container include radio-frequency identification (RFID) tags that track the number of injections (cartridge) and length of time on the instrument (cathode buffer container). This allows scientists, using the same instrument, to maintain custody of their own cartridges.

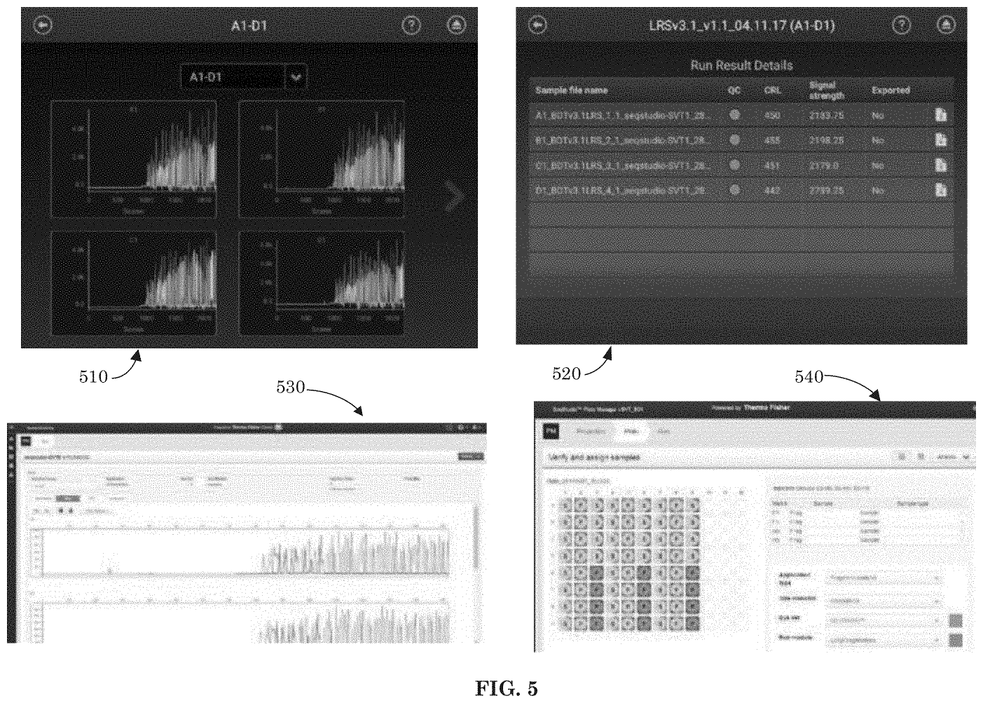

Genetic analyzer instrument system 300 allows real-time monitoring of runs on the SeqStudio Genetic Analyzer. As shown in FIG. 5, Instrument 300 displays results 510 for each capillary in real time. Once an injection is finished, several quality checks are calculated and displayed in exemplary screen display 520. If an injection produces poor traces or poor QC values, those samples can be re-injected, with altered injection parameters, if desired. Exemplary screenshot 530 from an off-site computer monitoring shows the progress of a run. In exemplary screenshot 540 as used in some embodiments of the present invention, runs set up in a PlateManager user interface can be uploaded directly to the instrument. PlateManager allows investigators to assign multiple sequencing and fragment analysis runs on the same plate, taking advantage of the universal polymer in the cartridge and use them only when needed, providing another level of flexibility. As shown in user interface screenshots 510-540 of FIG. 5, maintenance of the SeqStudio Genetic Analyzer used in some embodiments of the present invention is simple and straightforward for the user, and instrument calibrations used in the genetic analyzer instrument used in exemplary embodiments of the invention described herein may be handled automatically by leveraging advancements in imaging and algorithm tools.

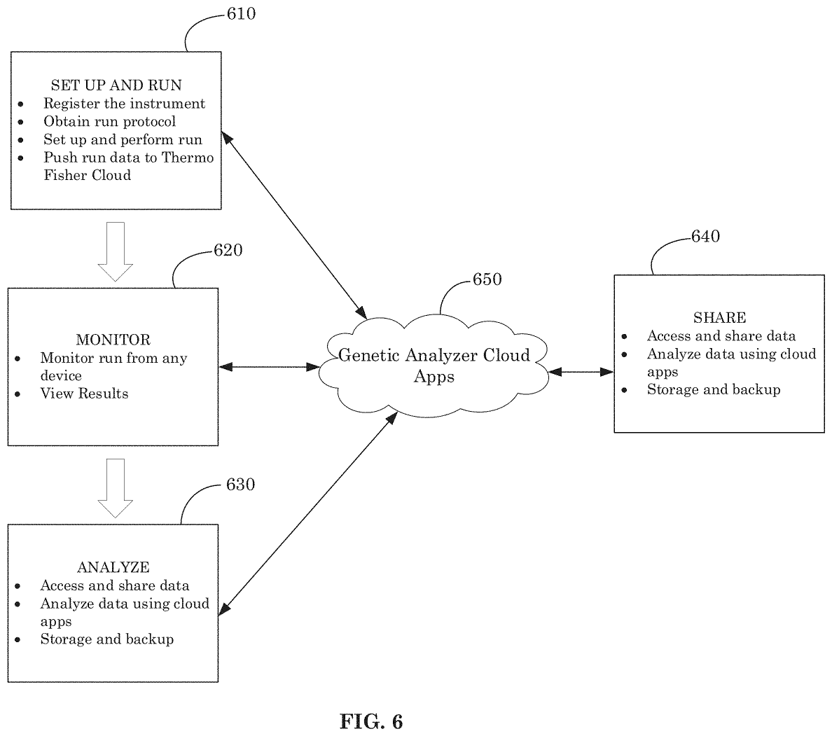

As shown in FIG. 6, another level of convenience may be added by integrating wireless connectivity into the exemplary genetic analyzer instrument shown in FIG. 3. FIG. 6 shows how the SeqStudio genetic analyzer instrument may be accessible to the user in several different ways: via the onboard interface, a remote computer, or a mobile device app. In some embodiments of the invention, assays or experimental runs can be set up using either the onboard computer or by using PlateManager, the stand-alone software that operates within Thermo Fisher Connect or on a separate computer, as shown in 610. By using web browser-based software, access to run setup, plate maps, run conditions, and analysis settings are all immediately available from anywhere you have Internet access. Injection conditions, reinjections, and reordering of injections can all be monitored and modified during the run as shown in 620, maximizing the ability to collect quality data from each plate. After data collection, the web browser-based suite of applications, including applications to measure microsatellite instability, allows accessible analysis in some embodiments of the present invention. Determination of DNA sequence variants, alignments, and fragment analysis are all available immediately upon completion of a run, in analysis step 630. Finally, the cloud connectivity 650 enables collaborators in different locations to monitor, access, share data information, and rapidly analyze the same data sets anytime post-run in sharing and collaboration step 640.

The SeqStudio Genetic Analyzer provides touchscreen usability via the instrument itself or via smartphone, tablet or other user device, allowing researches to collaborate and analyze data remotely as well as onsite with equal effectiveness. The exemplary genetic analyzer system discussed herein as used in some embodiments of the present invention is designed for both new and experienced users who need simple and affordable Sanger sequencing and fragment analysis, without compromising performance or quality.

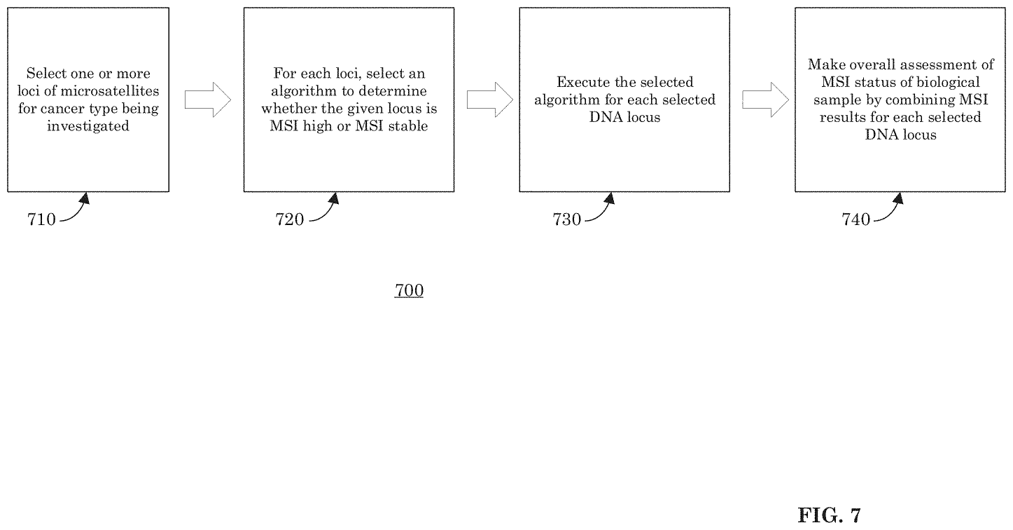

FIG. 7 shows a flow diagram of a method 700 used in some embodiments of the present invention to determine the MSI status of a biological sample. In step 710, one or more DNA loci of the biological sample are selected to investigate for tumors which exhibit a high mutation rate and hence a high microsatellite instability.

Microsatellite instability (MSI) is a form of genomic instability due to reduced fidelity during the replication of DNA; this is thought to be caused by defects in DNA repair mechanisms. Defects in this biomolecular machinery is most easily observed by examining places in the DNA where there is a single nucleotide (one of the four possible nucleotides) repeated many times (a homopolymer); e.g., GGGGGGGGGGG (SEQ ID NO: 32) is an 11-base repeat of Guanine. Extending this example, with damaged DNA repair mechanisms that often manifest in tumor cells, the section of DNA with the 11-base repeat of Guanine may be replicated as, for example, 10 bases or 5 bases or 13 bases instead of the normal 11 bases. Microsatellite instability analysis involves chemistries designed to examine several different regions in DNA at which there are homopolymers. These chemistries select out and amplify sections of DNA (an amplified fragment of DNA at specific DNA loci) that include each of the homopolymers of interest. Hence, again building on our 11-base Guanine example, normally, the amplified DNA at this locus would have a fragment size of, say, 20 bases (some number larger than 11 selected out by the chemistry). However, if DNA replication repair mechanisms are damaged, the replicated DNA may only have 10, for example, instead of the usual 11 Guanines so the amplified fragments will be of size 19 instead of 20. There are two ways to detect this situation using the technologies that are the subject of this disclosure: 1) analyze DNA from tumor tissue and normal tissue from the same person and compare the two or 2) analyze DNA from tumor tissue and compare to what is typically expected at each DNA locus of interest in the case where there is no damage to DNA repair mechanisms. Note that these concepts as well as the invention described in this disclosure also apply to non-homopolymer sections of DNA that consist of simple repeated sequences of nucleotides, e.g., ACACACAC or TATGTATGTATGTAGT (SEQ ID NO: 33), etc.

Thus, in step 710 particular DNA loci may be selected for the sensitivity of the loci to the cancer type under investigation as compared to other cancer types. A particular DNA locus (also referred to as a marker) may also be selected for the reliability of DNA amplification at that particular locus.

In step 720, each DNA locus is examined and one or more algorithms may be selected for each locus to determine whether that given locus is microsatellite unstable, MSU, or microsatellite stable, MSS. Embodiments of the present invention utilize a number of algorithms for determining whether or not a given DNA locus is MSU or MSS, including algorithms 1 through 11 below. In step 730, the selected algorithm(s) is executed for each selected DNA locus. In step 740, the overall MSI status is determined for the biological sample by combining the MSI results for each selected DNA locus.

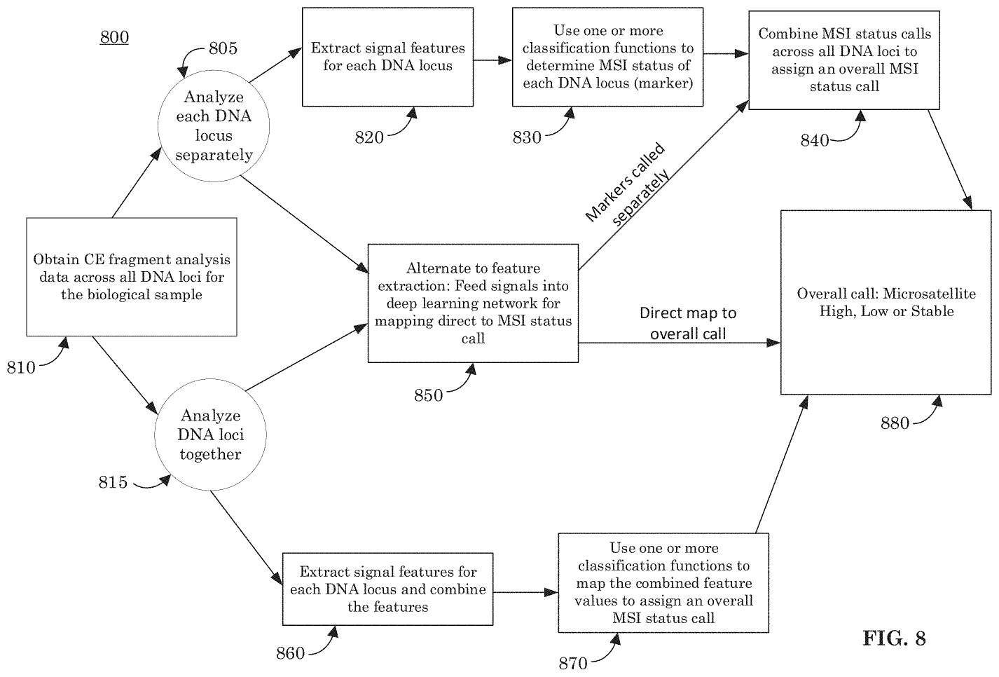

FIG. 8 shows another embodiment of a method for assessing the microsatellite instability of a biological sample, which is first described above and in FIG. 7. The embodiment shown in FIG. 8 provides additional detail and alternate data analysis pathways elaborating on the embodiment of the microsatellite instability assessment method shown in FIG. 7. Starting at step 810 of FIG. 8, the CE fragment analysis data across all DNA loci for the biological sample is obtained. Each DNA locus, or marker, may be analyzed separately, as shown in step 805. Alternatively, the markers may be analyzed together as shown in step 815 of FIG. 8. If the markers are to be analyzed separately as in step 805, one or more signal features can be extracted for each marker in step 820, and one or more classification functions such as the exemplary classification functions described below can be applied to the extracted signal features to determine the MSI status of each marker in step 830.

1. A simple size threshold: Any fluorescence peaks appearing below the fragment size threshold (or above depending on the location for an MSS situation) is considered MSU. This is appropriate if there is only one DNA locus covered by a given dye and the number of nucleotides differs significantly between MSU and MSS DNA molecule situations.

2. Fragment size interval: If there are any fluorescence peaks appearing within a given fragment size interval (an interval on the DNA fragment size axis of the data), the DNA locus is considered MSU. This presumes that there is no overlap between the size intervals covering all the DNA loci using the same dye and the fluorescent peaks associated with MSU and MSS situations are also well separated.

3. Peak count within a given size interval: If the number of significant fluorescent peaks (significance determined by peak size) is above a threshold, the locus is considered MSU. This presumes that there is no overlap between the size intervals covering all the DNA loci using the same dye.

4. Relative peak count within a given size interval: If the number of significant fluorescent peaks (significance determined by peak size) deviates significantly from that expected for MSS, the locus is considered MSU. This presumes that there is no overlap between the size intervals covering all the DNA loci using the same dye.

5. Peak envelope peaks within a given size interval: If the number of envelope peaks is two or more, the locus is considered MSI-high. This presumes that there is no overlap between the size intervals covering all the DNA loci using the same dye.

6. Peak envelope separation within a given size interval: If the separation between the two largest envelope peaks deviates significantly from that of MSI-stable samples, the locus is considered MSI-high. This presumes that there is no overlap between the size intervals covering all the DNA loci using the same dye.

7. Peak pattern: Peak patterns can consist of two or more values among the following: peak amplitudes and/or locations along the fragment size axis; peak amplitudes and/or locations relative to the largest peak; peak envelope peak amplitudes, locations, and/or widths; peak metrics relative to peak envelope metrics;

8. Peak pattern deviations from normal: Peak patterns of (7) above relative to these patterns from normal tissue samples;

9. Peak pattern deviations from normal (non-cancer) population peak patterns: Peak patterns of (7) above relative to these patterns from nominal values for these patterns, such as the mean, median, z-score, etc., across a population of people without cancer.

10. Peak pattern deviations from normal relative to population deviations: A combination of (8) and (9) above where the metrics of (8) are compared to nominal values of these patterns across a population of people without cancer.

11. Difference signal patterns: In the case that data from a given person is available from both normal and tumor tissue, signal patterns described above can be computed on the difference between normalized data from tumor and normal tissue. In addition, other metrics derived from the difference signal can be used to characterize the difference signal at each locus. For example, asymmetry of the difference signal can be characterized by the difference between the center of mass of the positive peaks of the difference signal compared to the negative peaks. Other examples include the relative position of the difference signal maximum and minimum, the root-mean-square (RMS) values of positive compared to negative peaks, overall RMS value for the difference signal, etc.

For items (7) through (11), the algorithm for determining whether a DNA locus is MSS or MSU would consist of a suitable classification function that can process multi-dimensional vectors. For example, discriminant functions, multi-layer artificial neural networks, vector machines, etc. are examples of typical machine learning methods that can be applied. Alternatively, instead of pre-specifying signal features as outlined in items (7) to (10), deep learning methods can be applied to automatically learn the best signal features to distinguish MSU from MSS by using a large number of samples of CE fragment analysis data localized to the fragment size intervals of interest.

To make the overall assessment of MSI status in step 740 of FIG. 7, or starting at step 810 of FIG. 8, the MSI results for each DNA locus can be combined across all of the markers in the nucleic acid sample under investigation to assign an overall MSI status call in step 840. The following are exemplary methods for doing this when two or more DNA loci are used:

12. Fixed percentage level: If the percentage of DNA loci that are MSU is above a chosen threshold, the overall assignment is MSI-high (or MSI-low if the percentage of DNA loci that are MSU are below the first chosen threshold but above a second predetermined threshold) and MSS if the percentage of DNA loci that are MSU are below both of these thresholds.

13. Weighted sum: In one embodiment of the invention, a weighted sum across DNA loci can be calculated after assigning MSU loci an exemplary value of 1 and MSS loci an exemplary value of 0; the overall assessment can be assigned MSI-high if the weighted sum across loci exceeds a threshold. Linear discriminant functions are an exemplary way to determine the weightings.

14. Non-linear classification: As shown in FIG. 8 at steps 820 and 830, the MSI results across DNA loci can alternatively be combined in a non-linear fashion to determine an overall assessment of MSI status in step 840. An exemplary way to determine the non-linear classification function is to train a multi-layer artificial neural network to make the assignment. Standard 3-layer artificial neural networks, trained with customary backpropagation techniques known in the art to minimize cross entropy, have been found to provide adequate accuracy in distinguishing MSU from MSS cases.

15. Direct to overall assessment: Instead of pre-assessing each DNA locus for MSI status as in step 805, the markers may be analyzed together in step 815 and the signal features expressed in items (7) to (11) can be combined across DNA loci as shown in step 860 and used to generate one or more classification functions that directly assigns the overall MSI status as shown in step 870 of FIG. 8. For example, suppose marker A used 3 signal features to determine whether it is MSU or MSS and marker B used 4 features for this. In one embodiment of the present invention, the features from both markers can be combined into a 7-dimensional feature vector to feed into an artificial intelligence generated classifier, such as an artificial neural network trained to map this vector to an overall MSI status call. Instead of an explicit algorithm to combine information across markers the combining of this information becomes inherent in the training of this artificial neural network classifier. Alternatively, artificial intelligence generated classifiers may be implemented via deep learning methods, which can be used as described above except that data across DNA loci are combined in the analysis either after the markers are analyzed separately in step 805 or together, as shown in step 815. The signal features are fed directly into a deep learning neural network for mapping directly into an MSI status call, as shown in step 850 of FIG. 8. For example, signals from each locus can be concatenated into one large signal vector and fed into a deep learning network to map them into an overall MSI status call at step 880.

Some embodiments of the present invention comprise methods for using one or more anti-tumor drugs to treat tumor patients. In particular embodiments, one or more of the methods, computer program products, systems, or kits disclosed herein are used to determine microsatellite instability of tumor cells in a biological sample obtained from a patient. Then, if microsatellite instability is determined to be high, the one or more anti-tumor drugs are administered to the patient to treat the tumor.

Systems, apparatus, and methods described herein may be implemented using a computer program product tangibly embodied in an information carrier, e.g., in a non-transitory machine-readable storage device, for execution by a programmable processor; and the method steps described herein, including one or more of the steps of the methods in FIG. 6, FIG. 7 and FIG. 8 and alternative embodiments may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

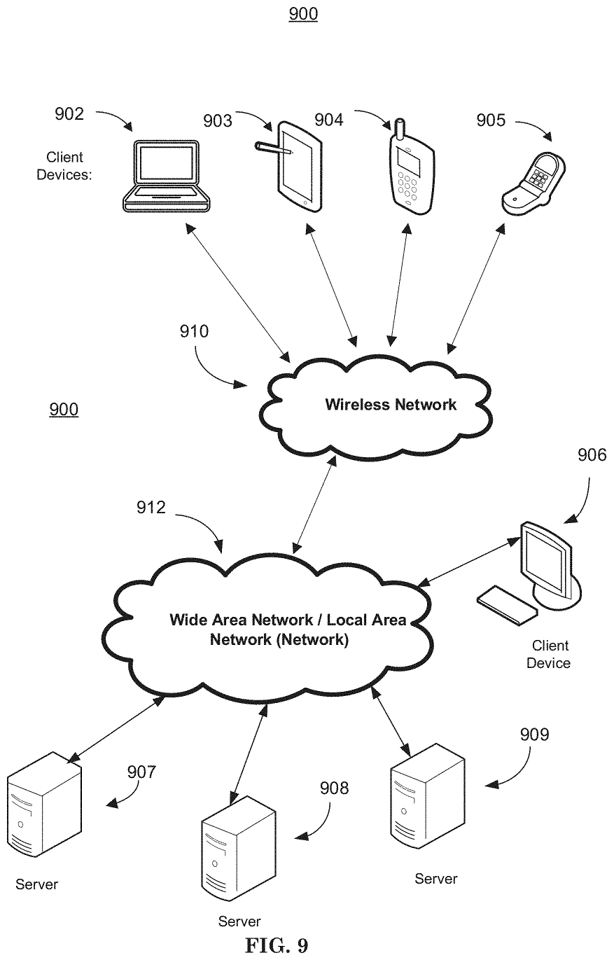

FIG. 9 illustrates components of one embodiment of an environment 900 in which the invention may be practiced. Not all the components may be required to practice the invention, and variations in the arrangement and type of the components may be made without departing from the spirit or scope of the invention. As shown, the system 900 includes one or more Local Area Networks (“LANs”)/Wide Area Networks (“WANs”) 912, one or more wireless networks 910, one or more wired or wireless client devices 906, mobile or other wireless client devices 902-906, servers 907-909, and may include or communicate with one or more data stores or databases. Various of the client devices 902-906 may include, for example, desktop computers, laptop computers, set top boxes, tablets, monitors, cell phones, smart phones, devices for interfacing with, or viewing dashboards or analytics relating to, genetic analysis related systems or entities, etc. The servers 907-909 can include, for example, one or more application servers, content servers, search servers, database servers, database management or SQL servers, other servers relating to genetic analysis related systems, etc.

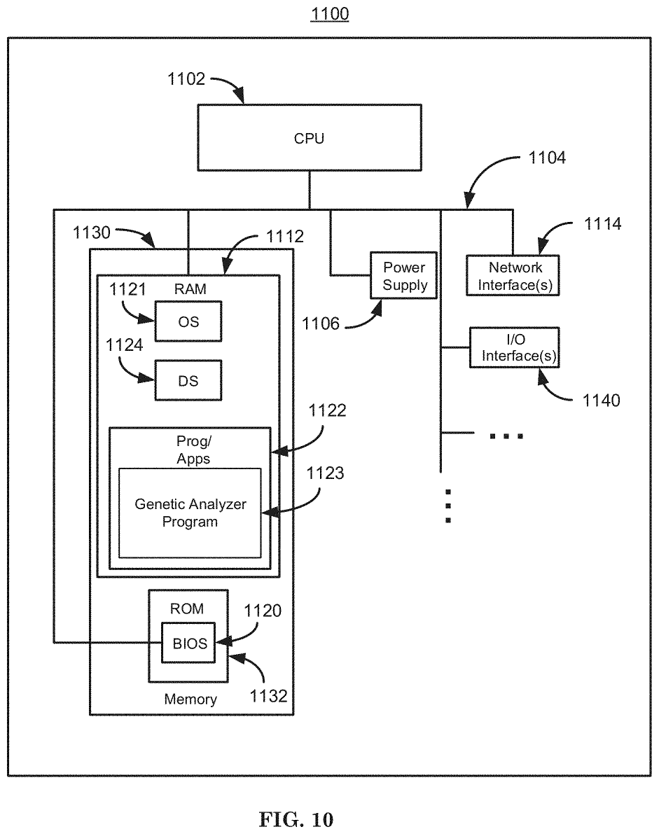

FIG. 10 illustrates a block diagram of an electronic device 1100 that can implement one or more aspects of genetic analysis related systems and methods according to embodiments of the invention. Instances of the electronic device 1100 may include servers, e.g., servers 907-909, and client devices, e.g., client devices 902-906.

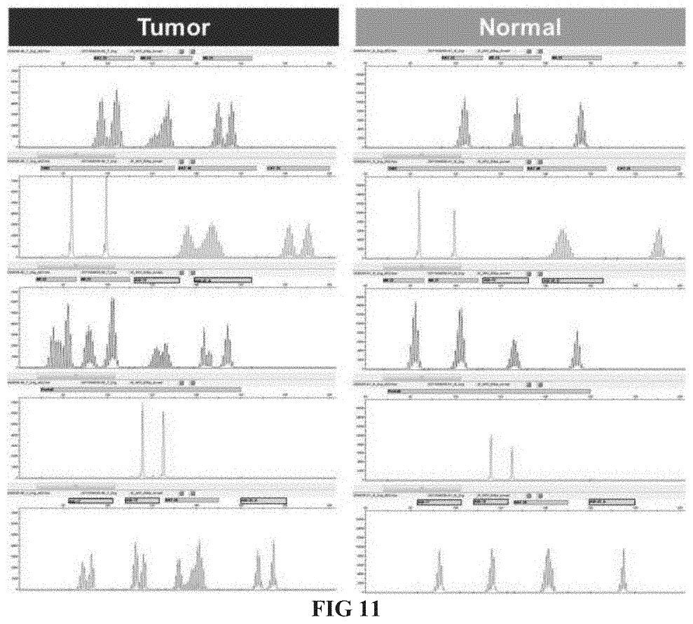

FIG. 11 shows an example of a computer system 1100, one or more of which may provide one or more of the components of, or alternatives to computer 103 of FIG. 1. Computer system 1100 executes instruction code contained in a computer program product 1122 comprising genetic analyzer program 1123 (which may, for example, comprise CE data analyzer program 104 of the computer program product 102 of the embodiment of FIG. 1.) Computer program product 1122 comprises executable code in an electronically readable medium that may instruct one or more computers such as computer system 1100 to perform processing that accomplishes the exemplary method steps performed by the embodiments referenced herein. The electronically readable medium may be any non-transitory medium that stores information electronically and may be accessed locally or remotely, for example via a network connection. In alternative embodiments, the medium may be transitory. The medium may include a plurality of geographically dispersed media each configured to store different parts of the executable code at different locations and/or at different times. The executable instruction code in an electronically readable medium directs the illustrated computer system 1100 to carry out various exemplary tasks described herein. The executable code for directing the carrying out of tasks described herein would be typically realized in software. However, it will be appreciated by those skilled in the art, that computers or other electronic devices might utilize code realized in hardware to perform many or all the identified tasks without departing from the present invention. Those skilled in the art will understand that many variations on executable code may be found that implement exemplary methods within the spirit and the scope of the present invention.

The code or a copy of the code contained in computer program product 1100 may reside in one or more storage persistent media (not separately shown) communicatively coupled to system 1100 for loading and storage in persistent storage device. In general, the electronic device 1100 can include a processor/CPU 1102, memory 1130, a power supply 1106, and input/output (I/O) components/devices 1140, e.g., microphones, speakers, displays, touchscreens, keyboards, mice, keypads, microscopes, GPS components, etc., which may be operable, for example, to provide graphical user interfaces, dashboards, etc.

A user may provide input via a touchscreen of an electronic device 1100. A touchscreen may determine whether a user is providing input by, for example, determining whether the user is touching the touchscreen with a part of the user's body such as his or her fingers. The electronic device 1100 can also include a communications bus 1104 that connects the aforementioned elements of the electronic device 1100. Network interfaces 1114 can include a receiver and a transmitter (or transceiver), and one or more antennas for wireless communications.

The processor 1102 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 1130, which can include Random Access Memory (RAM) 1112 and Read Only Memory (ROM) 1132, can be enabled by one or more of any type of memory device, e.g., a primary (directly accessible by the CPU) or secondary (indirectly accessible by the CPU) storage device (e.g., flash memory, magnetic disk, optical disk, and the like). The ROM 1132 can also include Basic Input/Output System (BIOS) 1120 of the electronic device.

The RAM can include an operating system 1121, data storage 1124, which may include one or more databases, and programs and/or applications 1122 and a genetic analyzer program 1123. The genetic analyzer program 1123 is intended to broadly include all programming, applications, algorithms, software and other and tools necessary to implement or facilitate methods and systems according to embodiments of the invention. Elements of the genetic analyzer program 1123 program may exist on a single server computer or be distributed among multiple computers, servers, devices or entities, or sites. Moreover, those skilled in the art will appreciate that in addition to storing computer program product 1122 for carrying out processing described herein, memory 1130 may be configured to store the various data elements referenced and illustrated herein.

The power supply 1106 contains one or more power components and facilitates supply and management of power to the electronic device 1100.

The input/output components, including Input/Output (I/O) interfaces 1140, can include, for example, any interfaces for facilitating communication between any components of the electronic device 1100, components of external devices (e.g., components of other devices of the network or system 1100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 1140 and the bus 1104 can facilitate communication between components of the electronic device 1100, and in an example can ease processing performed by the processor 1102.

Where the electronic device 1100 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications.

Any computing device capable of sending, receiving, and processing data over a wired and/or a wireless network may act as a server, such as in facilitating aspects of implementations of genetic analyzer related systems and methods according to embodiments of the invention. Devices acting as a server may include devices such as dedicated rack-mounted servers, desktop computers, laptop computers, set top boxes, integrated devices combining one or more of the preceding devices, etc.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of systems and methods according to embodiments of the invention. One or more servers may, for example, be used in hosting a Web site utilized in embodiments of the present invention. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of systems and methods according to embodiments of the invention. Content may include, for example, text, images, audio, video, and the like.

In example aspects of genetic analyzer systems and methods according to embodiments of the invention, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, monitors, sensor-equipped devices, laptop computers, set top boxes, wearable computers, integrated devices combining one or more of the preceding devices, and the like.

Client devices may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, GPS or other location-aware capability, and a 2D or 3D touch-sensitive color screen on which both text and graphics may be displayed.

Client devices, such as client devices 1002-1006, for example, as may be used in example systems and methods according to embodiments of the invention, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as viewing or interacting with analytics or dashboards, interacting with genetic analyzer instruments, methods or systems used in embodiments of the present invention, browsing webpages, using a web search engine, interacting with various apps stored on a smart phone, sending and receiving messages via email, SMS, or MMS, playing games, receiving advertising, watching locally stored or streamed video, or participating in social networks. In example aspects of genetic analyzer systems and methods according to embodiments of the invention, one or more networks, such as networks 1010 or 1012, for example, may couple servers and client devices with other computing devices, including through wireless network to client devices. A network may be enabled to employ any form of computer readable media for communicating information from one electronic device to another. A network may include the Internet in addition to Local Area Networks (LANs), Wide Area Networks (WANs), direct connections, such as through a Universal Serial Bus (USB) port, other forms of computer-readable media, or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 1010, as in example genetic analysis related systems and methods according to embodiments of the invention, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, 5G and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, AppleTalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in size), padding (may be composed of all zeros and includes a number of bits such that the header ends on a 32 bit boundary). The number of bits for each of the above may also be higher or lower.

A “content delivery network” or “content distribution network” (CDN), as may be used in example systems and methods according to embodiments of the invention, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's Web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

One embodiment of the present invention includes systems, methods, and a non-transitory computer readable storage medium or media tangibly storing computer program logic capable of being executed by a computer processor.

Those skilled in the art will appreciate computer system 1100 illustrates just one example of a system in which a computer program product in accordance with an embodiment of the present invention may be implemented. To cite but one example of an alternative embodiment, execution of instructions contained in a computer program product in accordance with an embodiment of the present invention may be distributed over multiple computers, such as, for example, over the computers of a distributed computing network.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

One skilled in the art would understand that descriptions of making and using the particles described herein is for the sole purpose of illustration, and that the present disclosure is not limited by this illustration.

### Example 1. DNA Isolation and Microsatellite Instability Analysis

Formalin-fixed, paraffin-embedded (FFPE) tissue sections (10 μm) were harvested from FFPE blocks using a Microm HM 355S rotary microtome. DNA was isolated from the FFPE sections using the RecoverAll™ Total Nucleic Acid Isolation Kit and the concentrations were measured on an Invitrogen Qubit 4 Fluorimeter using the Qubit™ dsDNA HS Assay Kit. The extracted FFPE DNA was diluted to 1 ng/μl in 1× low-EDTA TE buffer. Microsatellite instability in the tissue samples were assessed using the ABI MSI assay. The ABI MSI amplification mix consists of 4 μl of Multiplex PCR MasterMix, 4 μl of 1× low-EDTA TE buffer, and 2 μl of DNA at 1 ng/μl. The PCR was carried out in an Applied Biosystems ProFlex PCR system in GeneAmp™ PCR System 9700 simulation mode under the following conditions: 95° C. for 11 minutes, 29 cycles of denaturation at 94° C. for 20 seconds and annealing at 59° C. for 2 minutes; and a final extension at 60° C. for 25 minutes. Amplified PCR products were denatured for 95° C. for 3 minutes in a 20 μl reaction volume consisting of 17 μl of Hi-Di™ Formamide, 1 μl of GeneScan™ 600 LIZ™ dye Size Standard v2.0, and 2 μl of PCR product. Denatured amplified PCR products were then analyzed by fragment analysis on an ABI 3500 (xL) or SegStudio™ Genetic Analyzer.

PCR primers, including associated labeled, are shown in Table 1. PCR primers were labeled with the indicated dye at the 5′ end of the forward primer in each set, with the exception of ABI-16, which was labeled at the 5′ end of the reverse primer. The label was attached via a C3 linker.

TABLE 1
Primers for ABI-MSI Assay Amplification

| | Genome | | | | | |
|---|---|---|---|---|---|---|
| | Position | | | | | Amp |
| Marker | hg38 | Label | Forward | Reverse | Reverse2 | Length |
| BAT25. | chr4:5473189 | FAM | GGAGTGATT | TGACATTC | NA | 109 |
| 109bp | 5-54732210 | | CTCTAAAGA | TGCATTTT | | |
| | | | GTTTTGTGT | AACTATGG | | |
| | | | T | CT | | |
| | | | (SEQ ID | (SEQ ID | | |
| | | | NO. 1) | NO. 2) | | |
| BAT26. | chr2:4741427 | SID | TGAAATTGG | GCTCCTTT | NA | 141 |
| 170s.1 | 0-47414597 | | ATATTGCAG | ATAAGCTT | | |
| | | | CAGTCAGA | CTTCAGTA | | |
| | | | (SEQ ID | TATGTCA | | |
| | | | NO. 3) | (SEQ ID | | |
| | | | | NO. 4) | | |
| BAT40. | chr1:1195105 | VIC | CATTTTATA | GGGTGGTA | NA | 150 |
| nodeg | 67-119510904 | | TCCTCAAGC | GAGCAAGA | | |
| | | | CAAGATTAA | CC(SEQ ID | | |
| | | | CTT | NO. 6) | | |
| | | | (SEQ ID | | | |
| | | | NO. 5) | | | |
| CAT25. | chr7:1433060 | VIC | CCTGCTTAT | CCTGTAGTC | NA | 193 |
| 190s. | 99-143306424 | | CTGAAACTT | CCAGCTACT | | |
| 444 | | | CCCAACTT | TGGA | | |
| | | | (SEQ ID | (SEQ ID | | |
| | | | NO. 7) | NO. 8) | | |
| ABI-16 | chr17:509302 | SID | CTCCTCTGT | GCCACTGC | NA | 115 |
| | 5-5093343 | | CCTCCCACT | ATCCCATC | | |
| | | | GA | CT | | |
| | | | (SEQ ID | (SEQ ID | | |
| | | | NO. 9) | NO. 10) | | |
| ABI-17 | chr17:140777 | SID | AGGGAGGCT | CCTTGAAT | NA | 90 |
| | 72-14078083 | | TTTGAGAGC | TTCAGGCT | | |
| | | | AG | CAAGTCTC | | |
| | | | (SEQ ID | T | | |
| | | | NO. 11) | (SEQ ID | | |
| | | | | NO. 12) | | |
| ABI-19 | chr1:2353440 | NED | TAGTTTCTA | TTTCTAAG | NA | 132 |
| | 01-235344320 | | CACCCAAGC | GGAAACAT | | |
| | | | CACTGA | AAAACTTT | | |
| | | | (SEQ ID | CATTTTGG | | |
| | | | NO. 13) | (SEQ ID | | |
| | | | | NO. 14) | | |
| ABI- | chr12:111950 | SID | CTCTTTCAC | GCTTCGTC | NA | 172 |
| 20A | 319-111950639 | | TTGGCAGAA | GAAGATCA | | |
| | | | CATTG | GATAGTTG | | |
| | | | (SEQ ID | (SEQ ID | | |
| | | | NO. 15) | NO. 16) | | |
| ABI- | chr1:1516177 | NED | CAATGAATG | GGATCACT | NA | 158 |
| 20B | 28-151618041 | | GCAACCAGA | TGAGCCCA | | |
| | | | ATTAAATCC | GAATTCAA | | |
| | | | A | (SEQ ID | | |
| | | | (SEQ ID | NO. 18) | | |
| | | | NO. 17) | | | |
| NR21.3 | chr14:231829 | FAM | CTTTCTGG | CCATCCT | NA | 161 |
| 20s.1 | 87-23183298 | | TCACTCGC | GGTTTCT | | |
| | | | GTTTAC | GAAGACA | | |
| | | | (SEQ ID | CA | | |
| | | | NO. 19) | (SEQ ID | | |
| | | | | NO. 20) | | |
| NR22. | chr11:125620 | NED | CACTGAGC | GCCATCC | NA | 86 |
| nodeg | 720- | | ACATCACA | AGTTTTG | | |
| | 125621041 | | TTTAGGA | TTCTTAC | | |
| | | | (SEQ ID | AAAC | | |
| | | | NO. 21) | (SEQ ID | | |
| | | | | NO. 22) | | |
| NR24.2 | chr2:9518346 | FAM | GCTGAATT | CGGAGATT | NA | 133 |
| 40s.1 | 3-95183776 | | TTACCTCC | GTGCCATT | | |
| | | | TGACTCCA | GCATT | | |
| | | | A(SEQ ID | (SEQ ID | | |
| | | | NO. 23) | NO. 24) | | |
| NR27.2 | chr11:102322 | NED | CATGCTTG | CCATTAGT | NA | 109 |
| 20s.1 | 627- | | CAAACCAC | AAAGAGGT | | |
| | 102322953 | | TGGTAAAA | TCTGAGTC | | |
| | | | (SEQ ID | GAT | | |
| | | | NO. 25) | (SEQ ID | | |
| | | | | NO. 26) | | |
| PentaD. | chr21:436361 | TAZ | GAGCAAGA | GTGTATGA | GTGTAT | 131 |
| VF | 72-43636302 | | CACCATCT | TTCTCTTT | GATTCT | |
| | | | CAAGAAAG | TTTTCCCC | CTTTTT | |
| | | | (SEQ ID | TTC | TTCCCC | |
| | | | NO. 27) | (SEQ ID | TTT | |
| | | | | NO. 28) | (SEQ ID | |
| | | | | | NO. 29) | |
| TH01.92 | chr11:217104 | VIC | CTTCCGAG | GGCCTGT | NA | 92 |
| bp | 8-2171139 | | TGCAGGTC | TCCTCCC | | |
| | | | AC | TTATTT | | |
| | | | (SEQ ID | (SEQ ID | | |
| | | | NO. 30) | NO. 31) | | |

### Example 2. Assessment of MSI in Colorectal Cancer Versus Normal Tissue

DNA was isolated from FFPE sections of colorectal carcinoma (“Tumor”) or normal tissue (“Normal”) and PCR was performed as described in Example 1. Normal tissue is generally a matched normal control (tissue from the same patient). The signals in the electropherograms of FIG. 11 show clear differences between the signals of tumor versus normal samples. Signals are clear and easily interpretable with consistent signal strength.

### Example 3. Comparison of ABI-MSI with Other MSI Assays

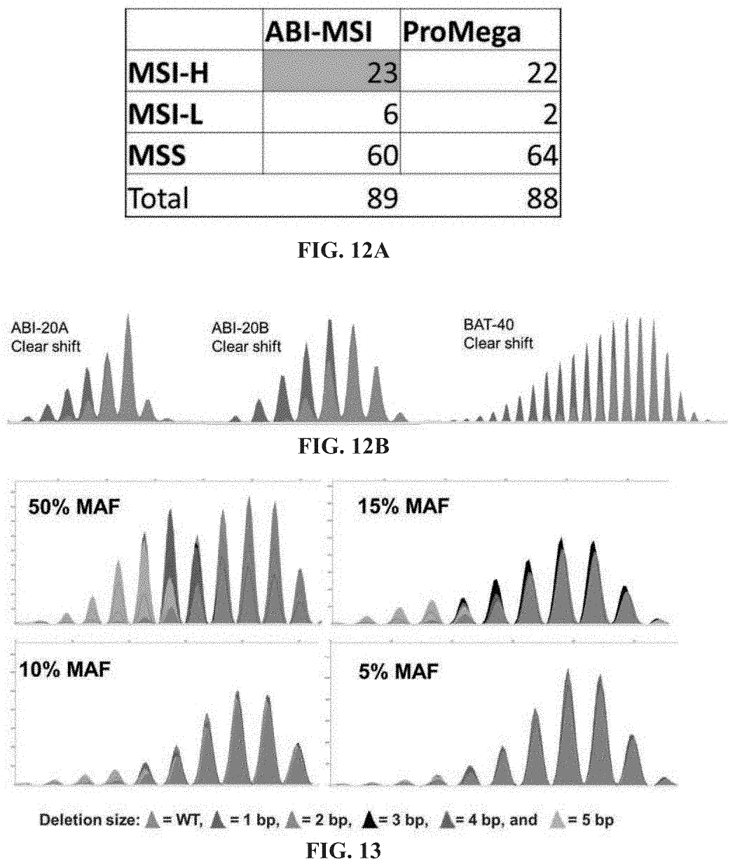

Endometrial carcinomas are notoriously difficult to assay, due to small deletions that are hard to resolve by standard MSI assays. DNA samples from endometrial carcinomas were amplified and analyzed as described in Example 1 (“ABI-MSI”), or using the PROMEGA™ MSI Analysis System using the protocol provided by the manufacturer. The PROMEGA™ system amplifies DNA from 5 loci (NR21, BAT26, BAT25, NR24, and MONO27), and includes two controls (PentaC and PentaD). As shown in FIG. 12A, the ABI-MSI assay identified one sample as MSI-high (MSI-H) that was identified as MSI-low (MSI-L) by the PROMEGA™ system, and identified five samples as MSI-low that were identified as MSI-stable (MSS) by the PROMEGA™ system. FIG. 12B shows clear shifts for several loci between normal (blue) and tumor (green) samples.

### Example 4. Development of an Expanded Microsatellite Instability Panel with Automated Data Analysis

In 2017, the Food and Drug Administration (FDA) approved pembrolizumab for any patients with solid tumors harboring MSI or mismatch repair deficiency. This has led to increased research utilizing MSI as a predictive biomarker for the effectiveness of immune-checkpoint inhibition. However, current solutions to detect MSI are few and have limitations, including insufficient markers for applications across multiple tumor types and cumbersome data analysis.

To improve upon the standard MSI detection panel and standard workflow, a MSI assay has been developed that has a fast, simple workflow, low sample input (2 ng FFPE DNA), expanded content, automated analysis and interpretable results, and tumor-only analysis. The assay takes only 3.5 hours from DNA to answer: 15 minutes of PCR sample preparation, 2 hours of fluorescent PCR, 15 minutes of fragment analysis sample preparation, 55 minutes of CE-based fragment analysis, and 5 minutes of data analysis.

FIG. 13 shows how synthetic constructs reveal detection complexities. Detection of instability is a complex interplay between deletion size and mutant allele fraction present in a sample. Synthetic constructs were generated to: 1) understand the peak morphology of difficult to assess MSI samples, and 2) train the algorithm at various allele frequencies and with variable deletion sizes for each homopolymer.

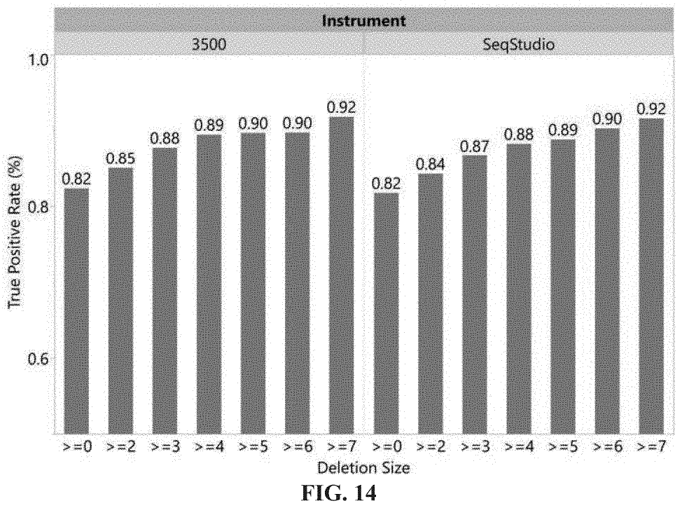

FIG. 14 shows tumor-only analysis at >98% specificity and >90% sensitivity at 5 bpd. Tumor-only analysis in cancer types with large deletions, like colon and gastric cancer, will see high sensitivity.

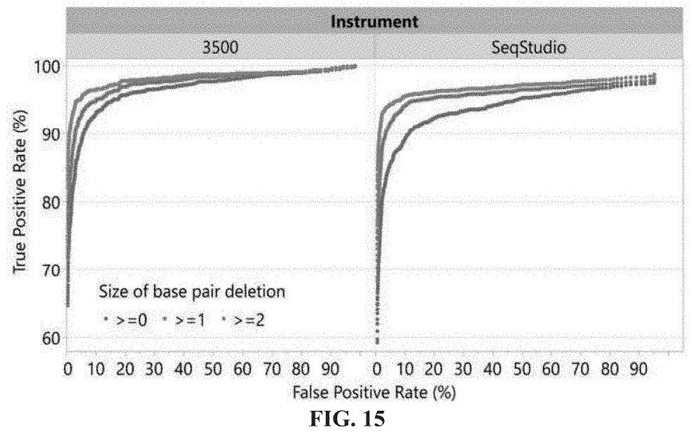

FIG. 15 shows tumor-normal analysis at >95% specificity and sensitivity at >3 bdp. Tunable algorithm parameters allow for maximization of sensitivity and specificity on both the Applied Biosystems™ 3500 and SeqStudio™ Genetic Analyzer Systems.

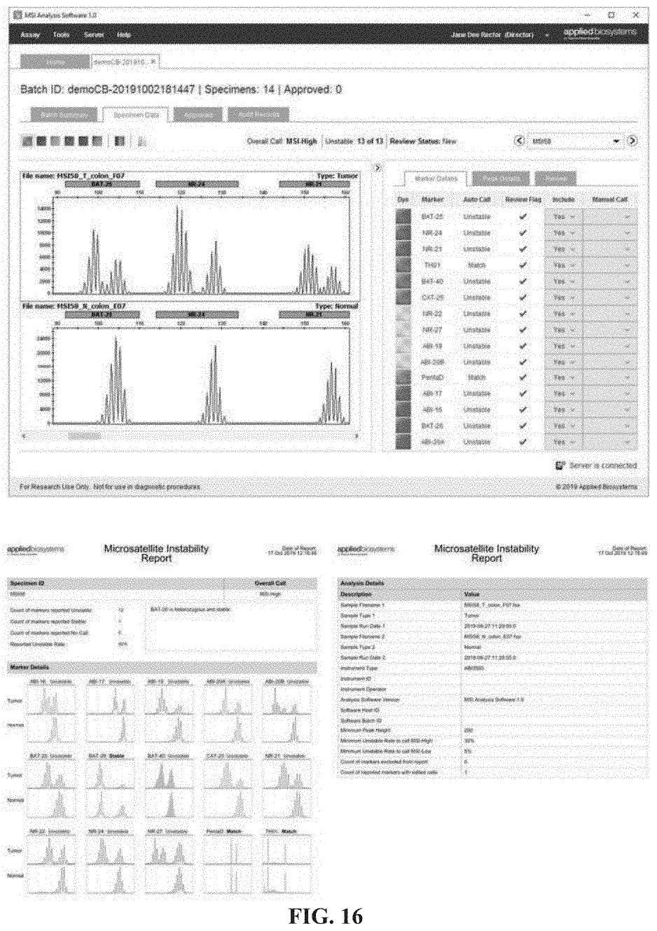

FIG. 16 illustrates the ABI MSI software (top) and example MSI report (bottom). The software provides an automated genotyping solution for streamlined analysis and reporting, saving customers time and effort required by current manual analysis.

The ABI MSI Assay achieves robust identification of microsatellite instability in multiple cancer types, with low sample input. In addition, the MSI analysis software has fast analysis and can include automated calling at sensitivity and specificity.

### REFERENCES

1. Vaksman, Zlaman and Harold R. Garner. “Somatic microsatellite variability as a predictive marker for colorectal cancer and liver cancer progression.” Oncotarget 6.8 (2015): 5760.
2. Le, Dung T., et al. “PD-1 blockade in tumors with mismatch-repair deficiency.” New England Journal of Medicine 372.26 (2015): 2509-2520.
3. Cortes-Ciriano, Isidro, et al. “A molecular portrait of microsatellite instability across multiple cancers.” Nature communications 8 (2017): 15180.
Latham et al, 2019

1. A method for detecting microsatellite instability (MSI) in a DNA sample, the method comprising:
a) co-amplifying a plurality of microsatellite loci of the DNA sample to produce amplified fragments comprising nucleic acid sequences from each locus, the plurality of loci comprising at least one locus selected from BAT25, BAT 26, BAT40, CAT25, NR21, NR22, NR24, NR27, ABI-20A, ABI-17, ABI-16, ABI-20B and ABI-19;
b) determining the size of the amplified fragments from each locus; and
c) comparing the size of the amplified fragments from each locus to the size of corresponding amplified fragments from a control, a difference in size between one or more amplified fragments between the sample and the control indicating the presence of MSI in the DNA sample.

2. A The method of claim 1 further comprising:
d) assigning a degree of MSI to the DNA sample, thereby determining the MSI status of the DNA sample.

3. A method for diagnosing cancerous tissue in a biological sample, the method comprising:
performing the method of claim 1; and
diagnosing the cancerous tissue in the presence of MSI in the biological sample.

4.-8. (canceled)

9. The method of claim 1 further comprising assigning whether the biological sample or DNA sample is microsatellite instability high, microsatellite instability low, or microsatellite stable.

10. The method of claim 1, wherein the DNA sample is assigned microsatellite instability high if more than about 30% of the loci amplified from the DNA sample are determined to have MSI.

11. The method of claim 1, wherein the DNA sample is assigned microsatellite instability low if less than about 30% but more than about 1% of the loci amplified from the DNA sample are determined to have MSI.

12. The method of claim 1, wherein the DNA sample is assigned microsatellite stable if none of the loci amplified from the DNA sample are determined to have MSI.

13. The method of claim 1, further comprising co-amplifying one or more identification markers in the co-amplifying step.

14. The method of claim 13, wherein the one or more identification markers comprise PENTAD and/or TH01.

15. The method of claim 1, wherein the plurality of loci comprise at least two loci selected from BAT25, BAT26, BAT40, CAT25, NR21, NR22, NR24, NR27, ABI-20A, ABI-17, ABI-16, ABI-20B and ABI-19.

16. The method of claim 15, wherein the plurality of loci comprise at least eight loci selected from BAT25, BAT26, BAT40, CAT25, NR21, NR22, NR24, NR27, ABI-20A, ABI-17, ABI-16, ABI-20B and ABI-19.

17. (canceled)

18. The method of claim 1, wherein the DNA sample is from tumor cells, cells suspected of being cancerous, or other biological material suspected of being cancerous.

19. (canceled)

20. The method of claim 1, wherein the microsatellite loci are co-amplified using one or more primers selected from SEQ ID NOS. 1-26.

21. The method of claim 20, wherein the microsatellite loci are co-amplified using a primer pair comprising a first primer and a second primer, wherein polynucleotide sequences of the first primer and the second primer comprise one of the following pairs: the polynucleotide sequence of SEQ ID NO.: 1 and the polynucleotide sequence of SEQ ID NO.: 2; the polynucleotide sequence of SEQ ID NO.: 3 and the polynucleotide sequence of SEQ ID NO.: 4; the polynucleotide sequence of SEQ ID NO.: 5 and the polynucleotide sequence of SEQ ID NO.: 6; the polynucleotide sequence of SEQ ID NO.: 7 and the polynucleotide sequence of SEQ ID NO.: 8; the polynucleotide sequence of SEQ ID NO.: 9 and the polynucleotide sequence of SEQ ID NO.: 10; the polynucleotide sequence of SEQ ID NO.: 11 and the polynucleotide sequence of SEQ ID NO.: 12; the polynucleotide sequence of SEQ ID NO.: 13 and the polynucleotide sequence of SEQ ID NO.: 14; the polynucleotide sequence of SEQ ID NO.: 15 and the polynucleotide sequence of SEQ ID NO.: 16; the polynucleotide sequence of SEQ ID NO.: 17 and the polynucleotide sequence of SEQ ID NO.: 18; the polynucleotide sequence of SEQ ID NO.: 19 and the polynucleotide sequence of SEQ ID NO.: 20; the polynucleotide sequence of SEQ ID NO.: 21 and the polynucleotide sequence of SEQ ID NO.: 22; the polynucleotide sequence of SEQ ID NO.: 23 and the polynucleotide sequence of SEQ ID NO.: 24; and/or the polynucleotide sequence of SEQ ID NO.: 25 and the polynucleotide sequence of SEQ ID NO.: 26.

22.-27. (canceled)

28. The method of claim 1, wherein the DNA sample and the paired normal DNA sample are from the same individual.

29. The method of claim 1, wherein the paired normal DNA sample is a control DNA from a non-cancerous tissue.

30.-33. (canceled)

34. The method of claim 1, wherein each amplified fragment comprises a fluorescent label, and wherein determining the size of the amplified fragments from each locus comprises:
obtaining a plurality of signals by detecting fluorescence of the amplified fragments wherein each signal corresponds to amplified fragments from one of a plurality of different microsatellite loci; and
determining one or more signal features for each of the plurality of signals.

35. The method of claim 34, further comprising applying one or more classifiers to one or more of the signal features of the plurality of microsatellite loci to identify whether the biological sample is microsatellite instability high, microsatellite instability low, or microsatellite stable.

36. The method of claim 1, wherein each amplified fragment comprises a fluorescent label, and wherein determining the size of the amplified fragments from each locus comprises:
obtaining a plurality of signals by detecting fluorescence of the amplified fragments, the nucleic acid sequences corresponding to a plurality of different microsatellite loci wherein each signal corresponds to one of the plurality of different microsatellite loci.

37. (canceled)

38. A primer set comprising one or more primer pairs, each primer pair comprising a first primer and a second primer, wherein polynucleotide sequences of the first primer and the second primer comprise one of the following pairs: the polynucleotide sequence of SEQ ID NO.: 1 and the polynucleotide sequence of SEQ ID NO.: 2; the polynucleotide sequence of SEQ ID NO.: 3 and the polynucleotide sequence of SEQ ID NO.: 4; the polynucleotide sequence of SEQ ID NO.: 5 and the polynucleotide sequence of SEQ ID NO.: 6; the polynucleotide sequence of SEQ ID NO.: 7 and the polynucleotide sequence of SEQ ID NO.: 8; the polynucleotide sequence of SEQ ID NO.: 9 and the polynucleotide sequence of SEQ ID NO.: 10; the polynucleotide sequence of SEQ ID NO.: 11 and the polynucleotide sequence of SEQ ID NO.: 12; the polynucleotide sequence of SEQ ID NO.: 13 and the polynucleotide sequence of SEQ ID NO.: 14; the polynucleotide sequence of SEQ ID NO.: 15 and the polynucleotide sequence of SEQ ID NO.: 16; the polynucleotide sequence of SEQ ID NO.: 17 and the polynucleotide sequence of SEQ ID NO.: 18; the polynucleotide sequence of SEQ ID NO.: 19 and the polynucleotide sequence of SEQ ID NO.: 20; the polynucleotide sequence of SEQ ID NO.: 21 and the polynucleotide sequence of SEQ ID NO.: 22; the polynucleotide sequence of SEQ ID NO.: 23 and the polynucleotide sequence of SEQ ID NO.: 24; and/or the polynucleotide sequence of SEQ ID NO.: 25 and the polynucleotide sequence of SEQ ID NO.: 26.

39.-102. (canceled)
